(19) United States
(12) Patent Application Publication
(10) Pub. No.: US 2022/0118234 A1
(43) Pub. Date: Apr. 21, 2022

(54) EXPANDABLE DRUG DELIVERY PILL

(71) Applicant: Alma Therapeutics Ltd., Petach Tikva (IL)

(72) Inventors: Yossi Gross, Moshav Mazor (IL); Oz Cabiri, Hod HaSharon (IL); Lital Ben-Shitrit, Ashkelon (IL)

(73) Assignee: Alma Therapeutics Ltd., Petach Tikva (IL)

(21) Appl. No.: 17/268,507

(22) PCT Filed: Aug. 13, 2019
(86) PCT No.: PCT/IL2019/050906

§ 371 Date: Feb. 15, 2021

Related U.S. Application Data
(63) Continuation-in-part of application No. 16/103,420, filed on Aug. 14, 2018, now Pat. No. 10,675,248.

(51) Int. Cl. A61M 31/00

(52) U.S. Cl. CPC A61M 31/002

(57) ABSTRACT

An ingestible pill (1220, 1320) is provided including a medication-delivery device (1224, 1324), which is configured to deliver a medication (52) and includes a patch (1230, 1330) (a) having upper and lower surfaces (1232, 1234) that face in generally opposite directions, (b) disposed within an enteric coating (22) in a compressed shape (1242), (c) configured to assume an expanded shape (1244) after the enteric coating (22) dissolves. The patch (1230, 1330) includes a chamber (1270) defined by (a) a substantially water-impermeable and substantially gas-impermeable, elastic layer (1274) shaped so as to define at least one window (1276) therethrough, and (b) a liquid-permeable and substantially gas-impermeable wetting layer (1278), which entirely covers the at least one window (1276) and is sealed to the elastic layer (1274) around the at least one window (1276). The patch (1230, 1330) further includes, disposed within the chamber (1270), a gas-generating substance (1280) that produces gas upon contact with liquid. Other embodiments are also described.

### CROSS-REFERENCE TO RELATED APPLICATIONS

The present application claims priority from and is a continuation-in-part of U.S. application Ser. No. 16/103,420, filed Aug. 14, 2018, which is assigned to the assignee of the present application and incorporated herein by reference.

### FIELD OF THE INVENTION

The present invention generally relates to medical apparatus. Specifically, the present invention relates to an ingestible capsule for administering medication to a subject.

### BACKGROUND OF THE INVENTION

Medication is frequently stored in a capsule and administered to a subject who swallows the capsule. The medication passes through the intestinal wall and enters the blood of the subject.

U.S. Pat. No. 8,287,902 to Gross, which is incorporated herein by reference, describes an ingestible capsule for delivering medication to a subject. A capsule coating dissolves in a gastrointestinal tract of the subject. An inner core of the capsule has an outer surface associated therewith. The outer surface is disposed within the coating and expands when the coating dissolves. A medication is disposed on the outer surface, and the outer surface is configured such that the medication contacts an intestinal wall of the subject when the outer surface expands.

U.S. Pat. No. 9,492,396 to Gross, which is incorporated herein by reference, describes an ingestible pill includes a coating configured to dissolve in a small intestine; a core, which includes a medication-delivery element, which (a) has a compressed shape when disposed within the coating, and (b) is configured to assume, after the coating dissolves, an expanded shape; a medication; and a mucoadhesive. When unconstrained in the expanded shape, the medication-delivery element (a) is shaped so as to define first and second surfaces on opposite sides of the medication-delivery element, which have respective outer perimeters, which surround respective spaces of the respective surfaces, which spaces have respective greatest dimensions equal to between 2 and 10 cm, and each of which spaces has an area equal to at least 50% of the square of the greatest dimension; and (b) has an average thickness between the first and the second surfaces of less than 6 mm. Each of the medication and the mucoadhesive at least partially coats the first surface.

### SUMMARY OF THE APPLICATION

In some applications of the present invention, an ingestible pill is provided that comprises an enteric coating, a medication, and a medication-delivery device, which is configured to deliver the medication. The medication-delivery device comprises a patch (a) having upper and lower surfaces that face in generally opposite directions, (b) disposed within the enteric coating in a compressed shape, and (c) configured to assume, after the enteric coating dissolves, an expanded shape in which the patch has an outer perimeter.

In some applications, the patch comprises:
a chamber defined by (a) a substantially water-impermeable and substantially gas-impermeable, elastic layer shaped so as to define at least one window therethrough, and (b) a liquid-permeable and substantially gas-impermeable wetting layer, which entirely covers the at least one window and is sealed to the elastic layer around the at least one window; and
a gas-generating substance that produces gas upon contact with liquid, the gas-generating substance disposed within the chamber.

The wetting layer allows bodily fluids to pass into the chamber and contact the gas-generating substance, upon dissolving of the enteric coating in the small intestine.

The generation of the gas within the chamber promotes transition of the patch from the compressed shape to the expanded shape, and may help push the upper surface of the patch against an intestinal wall.

In some applications of the present invention, an ingestible pill is provided that comprises an enteric coating; and a medication-delivery device, which (a) is pliable, (b) is disposed within the enteric coating, having a compressed shape enabled by the pliability, and (c) is shaped so as to define (i) one or more medication chambers and (ii) one or more expansible chambers. The medication-delivery device further comprises (i) one or more outer surfaces, all of which are pliable; (ii) hollow medication-delivery needles, which are coupled to at least one of the one or more outer surfaces; and (iii) a medication, which is contained within the one or more medication chambers. The medication-delivery device is configured to assume, after the enteric coating dissolves, an expanded shape in which the hollow medication-delivery needles (a) are in fluid communication with the one or more medication chambers and (b) extend away from the medication-delivery device. The medication-delivery device is configured such that expansion of the one or more expansible chambers forces the medication from the one or more medication chambers and out of the medication-delivery device through the hollow medication-delivery needles.

There is therefore provided, in accordance with an Inventive Concept 1 of the present invention, an ingestible pill including:

an enteric coating;

a medication; and

a medication-delivery device, which is configured to deliver the medication and includes a patch (a) having upper and lower surfaces that face in generally opposite directions, (b) disposed within the enteric coating in a compressed shape, (c) configured to assume, after the enteric coating dissolves, an expanded shape in which the patch has an outer perimeter, and (d) including:
(i) a chamber defined by:
   a substantially water-impermeable and substantially gas-impermeable, elastic layer shaped so as to define at least one window therethrough, and
   a liquid-permeable and substantially gas-impermeable wetting layer, which entirely covers the at least one window and is sealed to the elastic layer around the at least one window; and
(ii) a gas-generating substance that produces gas upon contact with liquid, the gas-generating substance disposed within the chamber.
Inventive Concept 2. The ingestible pill according to Inventive Concept 1, wherein a greatest distance between the upper and the lower surfaces of the patch is less than 6 mm when the patch assumes the expanded shape and is unconstrained.
Inventive Concept 3. The ingestible pill according to Inventive Concept 1, wherein the gas-generating substance includes a powder.
Inventive Concept 4. The ingestible pill according to Inventive Concept 1, wherein the gas-generating substance includes sodium bicarbonate.
Inventive Concept 5. The ingestible pill according to Inventive Concept 1, wherein the gas-generating substance is disposed in a vicinity of the at least one window.
Inventive Concept 6. The ingestible pill according to Inventive Concept 1, wherein the wetting layer includes a material selected from the group consisting of: cellulose and biocellulose.
Inventive Concept 7. The ingestible pill according to Inventive Concept 1, wherein the patch is rolled, folded, or rolled and folded when disposed within the enteric coating in the compressed shape.
Inventive Concept 8. The ingestible pill according to Inventive Concept 1, wherein the patch can inscribe a circle having a diameter of between 2 and 10 cm when the patch assumes the expanded shape and is unconstrained.
Inventive Concept 9. The ingestible pill according to Inventive Concept 1, wherein the patch is configured such that the expanded shape is a swollen shape when the patch assumes the expanded shape and is unconstrained.
Inventive Concept 10. The ingestible pill according to Inventive Concept 9, wherein the patch is configured such that the expanded shape is a swollen disc shape when the patch assumes the expanded shape and is unconstrained.
Inventive Concept 11. The ingestible pill according to Inventive Concept 1, wherein the wetting layer is sealed to an internal surface of the elastic layer around the at least one window.
Inventive Concept 12. The ingestible pill according to Inventive Concept 1, wherein the wetting layer is sealed to an external surface of the elastic layer around the at least one window.
Inventive Concept 13. The ingestible pill according to any one of Inventive Concepts 1-12, wherein the patch is arranged within the enteric coating in the compressed shape, such that when the patch assumes the expanded shape after the enteric coating dissolves in a small intestine of a subject, the upper surface of the patch contacts an intestinal wall.
Inventive Concept 14. The ingestible pill according to Inventive Concept 13, wherein the at least one window is disposed on a lower side of the chamber.
Inventive Concept 15. The ingestible pill according to Inventive Concept 13, wherein the medication-delivery device is configured to release the medication such that at least 60% of the released medication is released at the upper surface of the patch.
Inventive Concept 16. The ingestible pill according to Inventive Concept 15, wherein the medication-delivery device is configured to release the medication such that at least 80% of the released medication is released at the upper surface of the patch.
Inventive Concept 17. The ingestible pill according to any one of Inventive Concepts 1-12, wherein the elastic layer includes a thermoplastic elastomer (TPE).
Inventive Concept 18. The ingestible pill according to Inventive Concept 17, wherein the TPE includes thermoplastic polyurethane (TPU).
Inventive Concept 19. The ingestible pill according to any one of Inventive Concepts 1-12, wherein the medication-delivery device further includes a porous solid foam disposed within the chamber.
Inventive Concept 20. The ingestible pill according to Inventive Concept 19, wherein the gas-generating substance is at least partially disposed in the porous solid foam.
Inventive Concept 21. The ingestible pill according to Inventive Concept 19, wherein the gas-generating substance is at least partially disposed between the porous solid foam and the wetting layer.
Inventive Concept 22. The ingestible pill according to Inventive Concept 19, wherein the porous solid foam is fixed to one or more portions of an internal surface of the elastic layer so as to inhibit expansion of the chamber upon production of the gas by the gas-generating substance.
Inventive Concept 23. The ingestible pill according to Inventive Concept 22, wherein the porous solid foam is fixed to at least first and second portions of the internal surface of the elastic layer that respectively define the upper and the lower surfaces of the patch.
Inventive Concept 24. The ingestible pill according to Inventive Concept 22, wherein the porous solid foam is integral with at least a portion of the elastic layer.
Inventive Concept 25. The ingestible pill according to any one of Inventive Concepts 1-12, wherein the medication is solid and is shaped as at least respective portions of a plurality of medication-needles.
Inventive Concept 26. The ingestible pill according to Inventive Concept 25, wherein the medication-needles further include respective non-medication support structures.
Inventive Concept 27. The ingestible pill according to Inventive Concept 25,

wherein the patch is arranged within the enteric coating in the compressed shape, such that when the patch assumes the expanded shape after the enteric coating dissolves in a small intestine of a subject, the upper surface of the patch contacts an intestinal wall,

wherein at least 60% of the medication-needles are coupled to the upper surface of the patch.
Inventive Concept 28. The ingestible pill according to Inventive Concept 27, wherein at least 80% of the medication-needles are coupled to the upper surface of the patch.
Inventive Concept 29. The ingestible pill according to Inventive Concept 27, wherein the at least one window is through the lower surface of the patch.
Inventive Concept 30. The ingestible pill according to Inventive Concept 25, wherein each of the medication-needles has a length of between 20 and 600 microns.
Inventive Concept 31. The ingestible pill according to Inventive Concept 25, wherein a greatest radius of the medication-needles is between 20 and 150 microns.
Inventive Concept 32. The ingestible pill according to any one of Inventive Concepts 1-12, wherein the medication-delivery device includes hollow medication-delivery needles, which are configured to deliver the medication.

There is further provided, in accordance with an Inventive Concept 33 of the present invention, an ingestible pill including:

an enteric coating;

a medication; and

a medication-delivery device, which is configured to deliver the medication and includes a patch having upper and lower surfaces that face in generally opposite directions, and an outer perimeter,

wherein the patch is disposed within the enteric coating in a compressed shape, in which two opposite perimeter portions of the outer perimeter are (a) rolled toward each other over the lower surface of the patch or (b) folded toward each other over the lower surface of the patch, such that, in either case, the two opposite perimeter portions touch the lower surface, and

wherein the patch is configured to assume an expanded shape after the enteric coating dissolves.
Inventive Concept 34. The ingestible pill according to Inventive Concept 33, wherein the patch is disposed within the enteric coating in the compressed shape, in which the two opposite perimeter portions of the outer perimeter are rolled toward each other over the lower surface of the patch.
Inventive Concept 35. The ingestible pill according to Inventive Concept 33, wherein the patch is disposed within the enteric coating in the compressed shape, in which the two opposite perimeter portions of the outer perimeter are folded toward each other over the lower surface of the patch.
Inventive Concept 36. The ingestible pill according to Inventive Concept 33, wherein the patch is disposed within the enteric coating in the compressed shape, in which the two opposite perimeter portions of the outer perimeter are (a) rolled at least one full turn toward each other over the lower surface of the patch or (b) folded at least one full turn toward each other over the lower surface of the patch.
Inventive Concept 37. The ingestible pill according to Inventive Concept 33, wherein the patch can inscribe a circle having a diameter of between 2 and 10 cm when the patch assumes the expanded shape and is unconstrained.
Inventive Concept 38. The ingestible pill according to Inventive Concept 33, wherein the patch is configured such that the expanded shape is a swollen shape when the patch assumes the expanded shape and is unconstrained.
Inventive Concept 39. The ingestible pill according to Inventive Concept 38, wherein the patch is configured such that the expanded shape is a swollen disc shape when the patch assumes the expanded shape and is unconstrained.
Inventive Concept 40. The ingestible pill according to Inventive Concept 33, wherein the patch is arranged within the enteric coating in the compressed shape, such that when the patch assumes the expanded shape after the enteric coating dissolves in a small intestine of a subject, the upper surface of the patch contacts an intestinal wall.
Inventive Concept 41. The ingestible pill according to any one of Inventive Concepts 33-40, wherein the patch is disposed within the enteric coating in the compressed shape, in which:

first, the two opposite perimeter portions of the outer perimeter are (a) rolled toward each other over the lower surface of the patch or (b) folded toward each other over the lower surface of the patch, such that, in either case, the rolled or folded patch has a length greater than a width thereof, and

then, the rolled or folded patch is folded widthwise.
Inventive Concept 42. The ingestible pill according to Inventive Concept 41, wherein the patch is disposed within the enteric coating in the compressed shape, in which:

first, the two opposite perimeter portions of the outer perimeter are (a) rolled toward each other over the lower surface of the patch or (b) folded toward each other over the lower surface of the patch, such that, in either case, the rolled or folded patch has the length greater than the width thereof, and

then, the rolled or folded patch is three-panel folded widthwise.
Inventive Concept 43. The ingestible pill according to Inventive Concept 42, wherein the rolled or folded patch is tri-folded.
Inventive Concept 44. The ingestible pill according to Inventive Concept 42, wherein the rolled or folded patch is three-panel gate folded.
Inventive Concept 45. The ingestible pill according to any one of Inventive Concepts 33-40, wherein the medication is solid and is shaped as at least respective portions of a plurality of medication-needles.
Inventive Concept 46. The ingestible pill according to Inventive Concept 45, wherein the medication-needles further include respective non-medication support structures.
Inventive Concept 47. The ingestible pill according to Inventive Concept 45,

wherein the patch is arranged within the enteric coating in the compressed shape, such that when the patch assumes the expanded shape after the enteric coating dissolves in a small intestine of a subject, the upper surface of the patch contacts an intestinal wall, and

wherein at least 60% of the medication-needles are coupled to the upper surface of the patch.

Inventive Concept 48. The ingestible pill according to Inventive Concept 47, wherein at least 80% of the medication-needles are coupled to the upper surface of the patch.
Inventive Concept 49. The ingestible pill according to Inventive Concept 45, wherein each of the medication-needles has a length of between 20 and 600 microns.
Inventive Concept 50. The ingestible pill according to Inventive Concept 45, wherein a greatest radius of the medication-needles is between 20 and 150 microns.
Inventive Concept 51. The ingestible pill according to any one of Inventive Concepts 33-40, wherein the medication-delivery device further includes hollow medication-delivery needles, which are configured to deliver the medication.

There is still further provided, in accordance with an Inventive Concept 52 of the present invention, an ingestible pill including:

an enteric coating;

a medication; and

a medication-delivery device, which is configured to deliver the medication and includes a patch having upper and lower surfaces that face in generally opposite directions,

wherein the patch is disposed within the enteric coating in a compressed shape, in which the patch is three-panel folded, and

wherein the patch is configured to assume, after the enteric coating dissolves, an expanded shape in which the patch has an outer perimeter.
Inventive Concept 53. The ingestible pill according to Inventive Concept 52, wherein the patch is tri-folded.
Inventive Concept 54. The ingestible pill according to Inventive Concept 52, wherein the patch is three-panel gate folded.
Inventive Concept 55. The ingestible pill according to Inventive Concept 52, wherein the patch can inscribe a circle having a diameter of between 2 and 10 cm when the patch assumes the expanded shape and is unconstrained.
Inventive Concept 56. The ingestible pill according to Inventive Concept 52, wherein the patch is configured such that the expanded shape is a swollen shape when the patch assumes the expanded shape and is unconstrained.
Inventive Concept 57. The ingestible pill according to Inventive Concept 56, wherein the patch is configured such that the expanded shape is a swollen disc shape when the patch assumes the expanded shape and is unconstrained.
Inventive Concept 58. The ingestible pill according to Inventive Concept 52, wherein the patch is arranged within the enteric coating in the compressed shape, such that when the patch assumes the expanded shape after the enteric coating dissolves in a small intestine of a subject, the upper surface of the patch contacts an intestinal wall.
Inventive Concept 59. The ingestible pill according to any one of Inventive Concepts 52-58, wherein the patch is disposed within the enteric coating in the compressed shape, in which:

first, the patch is rolled such that the rolled patch has a length greater than a width thereof, and

then, the rolled patch is three-panel folded widthwise.
Inventive Concept 60. The ingestible pill according to Inventive Concept 59, wherein the patch is disposed within the enteric coating in the compressed shape, in which:

first, the patch is rolled to form a single spiral in cross-section, and

then, the rolled patch is three-panel folded widthwise.
Inventive Concept 61. The ingestible pill according to Inventive Concept 59, wherein the patch is disposed within the enteric coating in the compressed shape, in which:

first, two opposite perimeter portions of the outer perimeter are rolled toward each other over the lower surface of the patch, and

then, the rolled patch is three-panel folded widthwise.
Inventive Concept 62. The ingestible pill according to any one of Inventive Concepts 52-58, wherein the patch is disposed within the enteric coating in the compressed shape, in which:

first, two opposite perimeter portions of the outer perimeter of the patch are folded toward each other over the lower surface of the patch, such that the folded patch has a length greater than a width thereof, and

then, the folded patch is folded widthwise.
Inventive Concept 63. The ingestible pill according to any one of Inventive Concepts 52-58, wherein the medication is solid and is shaped as at least respective portions of a plurality of medication-needles.
Inventive Concept 64. The ingestible pill according to Inventive Concept 63, wherein the medication-needles further include respective non-medication support structures.
Inventive Concept 65. The ingestible pill according to Inventive Concept 63, wherein at least 60% of the medication-needles are coupled to the upper surface of the patch.
Inventive Concept 66. The ingestible pill according to Inventive Concept 65, wherein at least 80% of the medication-needles are coupled to the upper surface of the patch.
Inventive Concept 67. The ingestible pill according to Inventive Concept 63, wherein each of the medication-needles has a length of between 20 and 600 microns.
Inventive Concept 68. The ingestible pill according to Inventive Concept 63, wherein a greatest radius of the medication-needles is between 20 and 150 microns.
Inventive Concept 69. The ingestible pill according to any one of Inventive Concepts 52-58, wherein the medication-delivery device further includes hollow medication-delivery needles, which are configured to deliver the medication.

There is additionally provided, in accordance with an Inventive Concept 70 of the present invention, a method including:

receiving, by a subject, an ingestible pill, which includes an enteric coating; a medication; and a medication-delivery device, which is configured to deliver the medication and includes a patch (a) having upper and lower surfaces that face in generally opposite directions, (b) disposed within the enteric coating in a compressed shape, and (c) including: (i) a chamber defined by: (A) a substantially water-impermeable and substantially gas-impermeable, elastic layer shaped so as to define the upper and at least one window therethrough, and (B) a liquid-permeable and substantially gas-impermeable wetting layer, which entirely covers the at least one window and is sealed to the elastic layer around the at least one window; and (ii) a gas-generating substance that produces gas upon contact with liquid, the gas-generating substance disposed within the chamber; and

swallowing the ingestible pill by the subject, so that the coating dissolves in a small intestine and the patch assumes an expanded shape, in which the patch has an outer perimeter.

There is yet additionally provided, in accordance with an Inventive Concept 71 of the present invention, a method including:

receiving, by a subject, an ingestible pill, which includes an enteric coating; a medication; and a medication-delivery device, which is configured to deliver the medication and includes a patch having upper and lower surfaces that face in generally opposite directions, and an outer perimeter, wherein the patch is disposed within the enteric coating in a compressed shape, in which two opposite perimeter portions of the outer perimeter are (a) rolled toward each other over the lower surface of the patch or (b) folded toward each other over the lower surface of the patch, such that, in either case, the two opposite perimeter portions touch the lower surface; and

swallowing the ingestible pill by the subject, so that the coating dissolves in a small intestine and the patch assumes an expanded shape.

There is also provided, in accordance with an Inventive Concept 72 of the present invention, a method including:

receiving, by a subject, an ingestible pill, which includes an enteric coating; a medication; and a medication-delivery device, which is configured to deliver the medication and includes a patch having upper and lower surfaces that face in generally opposite directions, wherein the patch is disposed within the enteric coating in a compressed shape, in which the patch is three-panel folded; and

swallowing the ingestible pill by the subject, so that the coating dissolves in a small intestine and the patch assumes an expanded shape in which the patch has an outer perimeter.

There is further provided, in accordance with an Inventive Concept 73 of the present invention, an ingestible pill including:

an enteric coating; and

a medication-delivery device, which includes (a) a patch having upper and lower surfaces that face in generally opposite directions; and (b) needles,

wherein the patch is disposed within the enteric coating, folded so as to define one or more creases, which define respective inner and outer crease sides, wherein at least 50% of the needles are coupled to the patch along the inner crease sides, and

wherein the patch is configured to assume, after the enteric coating dissolves, an expanded shape, in which the patch has an outer perimeter.
Inventive Concept 74. The ingestible pill according to Inventive Concept 73, wherein the patch can inscribe a circle having a diameter of between 2 and 10 cm when the patch assumes the expanded shape and is unconstrained.
Inventive Concept 75. The ingestible pill according to Inventive Concept 73, wherein each of the needles has a length of between 20 and 600 microns.
Inventive Concept 76. The ingestible pill according to Inventive Concept 73, wherein the patch is configured such that the expanded shape is generally flat when the medication-delivery device is unconstrained.
Inventive Concept 77. The ingestible pill according to Inventive Concept 73, wherein a greatest radius of the needles is between 20 and 150 microns.
Inventive Concept 78. The ingestible pill according to Inventive Concept 73, wherein the patch is annular.
Inventive Concept 79. The ingestible pill according to any one of Inventive Concepts 73-78, wherein between 0% and 10% of the needles are coupled to the patch along the outer crease sides when the patch is disposed within the enteric coating.
Inventive Concept 80. The ingestible pill according to any one of Inventive Concepts 73-78, wherein the ingestible pill is configured such that when the patch assumes the expanded shape upon dissolving of the enteric coating in a small intestine of a subject, the upper surface of the patch contacts an intestinal wall, thereby bringing the needles into contact with the intestinal wall.
Inventive Concept 81. The ingestible pill according to Inventive Concept 80, wherein at least 50% of the needles are coupled to the upper surface of the patch along the inner crease sides when the patch is disposed within the enteric coating.
Inventive Concept 82. The ingestible pill according to Inventive Concept 81, wherein at least 80% of the needles are coupled to the upper surface of the patch when the patch is disposed within the enteric coating.
Inventive Concept 83. The ingestible pill according to Inventive Concept 80, wherein the patch is disposed within the enteric coating, folded such that when the patch assumes the expanded shape upon dissolving of the enteric coating in the small intestine, the upper face of the patch contacts the intestinal wall, thereby bringing the needles into contact with the intestinal wall.
Inventive Concept 84. The ingestible pill according to any one of Inventive Concepts 73-78, wherein the medication-delivery device further includes a medication, and wherein the needles are configured to deliver the medication.
Inventive Concept 85. The ingestible pill according to Inventive Concept 84, wherein the needles are hollow and are configured to deliver the medication.
Inventive Concept 86. The ingestible pill according to any one of Inventive Concepts 73-78, wherein the needles include a solid medication.
Inventive Concept 87. The ingestible pill according to any one of Inventive Concepts 73-78, wherein the patch is disposed within the enteric coating, folded so as to define a plurality of creases.
Inventive Concept 88. The ingestible pill according to Inventive Concept 87, wherein the patch is disposed within the enteric coating, folded first in half and then accordion-folded.
Inventive Concept 89. The ingestible pill according to Inventive Concept 87, wherein the needles are not coupled to respective inner crease sides of a portion of the creases.
Inventive Concept 90. The ingestible pill according to Inventive Concept 89, wherein the needles are not coupled to respective inner crease sides of two or more of the creases.

There is still further provided, in accordance with an Inventive Concept 91 of the present invention, an ingestible pill including:

an enteric coating; and

a medication-delivery device, which includes:
(a) a patch, which (i) has upper and lower surfaces that face in generally opposite directions, (ii) has a compressed shape when disposed within the enteric coating, (iii) is shaped so as to define an outer perimeter, and (iv) includes at least first and second layers, which are arranged so as to define one or more expansible chambers between the first layer and the second layer, wherein the first layer and the second layer are fixed together at fixation locations, wherein some of the fixation locations are located at or near the outer perimeter and some of the fixation locations are located at least 2 mm from the outer perimeter; and
(b) needles, which are coupled to the upper surface at respective needle locations,

wherein the patch is configured such that, if the patch were laid generally flat on a flat horizontal surface, at least 80% of the needle locations would be horizontally offset from the fixation locations, and

wherein the patch is configured to assume an expanded shape after the enteric coating dissolves.
Inventive Concept 92. The ingestible pill according to Inventive Concept 91, wherein the patch can inscribe a circle having a diameter of between 2 and 10 cm when the patch assumes the expanded shape and is unconstrained.
Inventive Concept 93. The ingestible pill according to Inventive Concept 91, wherein each of the needles has a length of between 20 and 600 microns.
Inventive Concept 94. The ingestible pill according to Inventive Concept 91, wherein the patch is configured such that, if the patch were laid generally flat on the flat horizontal surface, all of the needle locations would be horizontally offset from the fixation locations.
Inventive Concept 95. The ingestible pill according to Inventive Concept 91, wherein the patch is configured such that, if the patch were laid generally flat on the flat horizontal surface, each of the needle locations would be located at least 1 mm horizontally from a nearest one of the fixation locations.
Inventive Concept 96. The ingestible pill according to Inventive Concept 91, wherein the patch is configured such that, if the patch were laid generally flat on the flat horizontal surface, each of the needle locations would be located approximately horizontally centered between a nearest two of the fixation locations.
Inventive Concept 97. The ingestible pill according to Inventive Concept 91, wherein the patch is configured such that the expanded shape is generally flat when the medication-delivery device is unconstrained.
Inventive Concept 98. The ingestible pill according to Inventive Concept 91, wherein the needles include a solid medication.
Inventive Concept 99. The ingestible pill according to Inventive Concept 91, wherein at least some of the fixation locations are arranged in a plurality of points.
Inventive Concept 100. The ingestible pill according to Inventive Concept 91, wherein the patch is annular.
Inventive Concept 101. The ingestible pill according to any one of Inventive Concepts 91-100, wherein the patch is configured such that, if the patch were laid generally flat on the flat horizontal surface, the needle locations would be located on average at least 1 mm from respective nearest fixation locations.
Inventive Concept 102. The ingestible pill according to Inventive Concept 101, wherein the patch is configured such that, if the patch were laid generally flat on the flat horizontal surface, the needle locations would be located on average at least 2 mm horizontally from respective nearest fixation locations.
Inventive Concept 103. The ingestible pill according to any one of Inventive Concepts 91-100, wherein the medication-delivery device further includes a medication, and wherein the needles are configured to deliver the medication.
Inventive Concept 104. The ingestible pill according to Inventive Concept 103,

wherein the patch further includes a third layer, which defines the upper surface,

wherein the second layer and the third layer are arranged so as define one or more medication chambers between the second layer and the third layer, wherein the one or more medication chambers contain the medication, and

wherein the needles are hollow.
Inventive Concept 105. The ingestible pill according to Inventive Concept 103, wherein the medication is contained within the one or more expansible chambers, and wherein the needles are (a) hollow, (b) coupled to the second layer in fluid communication with the one or more expansible chambers, when the patch assumes the expanded shape, and (c) configured to deliver the medication.
Inventive Concept 106. The ingestible pill according to Inventive Concept 105, wherein the ingestible pill is configured such that when the patch assumes the expanded shape upon dissolving of the enteric coating in a small intestine of a subject, the upper surface of the patch contacts an intestinal wall, thereby bringing the needles into contact with the intestinal wall.
Inventive Concept 107. The ingestible pill according to any one of Inventive Concepts 91-100, wherein at least some of the fixation locations are arranged in a plurality of segments.
Inventive Concept 108. The ingestible pill according to Inventive Concept 107, wherein at least some of the segments are curved when the patch assumes the expanded shape.
Inventive Concept 109. The ingestible pill according to Inventive Concept 108, wherein at least some of the curved segments are arranged equidistantly around a center point of the patch when the patch assumes the expanded shape.
Inventive Concept 110. The ingestible pill according to any one of Inventive Concepts 91-100, wherein the one or more expansible chambers include one or more inflatable chambers.
Inventive Concept 111. The ingestible pill according to Inventive Concept 110, wherein the one or more inflatable chambers contain a substance that produces gas upon contact with liquid.
Inventive Concept 112. The ingestible pill according to any one of Inventive Concepts 91-100, wherein the one or more expansible chambers contain a substance that expands upon contact with liquid.
Inventive Concept 113. The ingestible pill according to Inventive Concept 112, wherein the substance includes a polymer.
Inventive Concept 114. The ingestible pill according to any one of Inventive Concepts 91-100, wherein the first layer is liquid-permeable and substantially not gas-permeable.
Inventive Concept 115. The ingestible pill according to Inventive Concept 114, wherein the first layer includes biocellulose.
Inventive Concept 116. The ingestible pill according to any one of Inventive Concepts 91-100, wherein the patch is disposed within the enteric coating, folded so as to define one or more creases, which define respective inner and outer crease sides, and wherein at least 50% of the needles are coupled to the upper surface of the patch along the inner crease sides.
Inventive Concept 117. The ingestible pill according to Inventive Concept 116, wherein the patch is disposed within the enteric coating, folded so as to define a plurality of creases.
Inventive Concept 118. The ingestible pill according to Inventive Concept 117, wherein the needles are not coupled to respective inner crease sides of a portion of the creases.
Inventive Concept 119. The ingestible pill according to Inventive Concept 116, wherein between 0% and 10% of the needles are coupled to the patch along the outer crease sides when the patch is disposed within the enteric coating.

There is additionally provided, in accordance with an Inventive Concept 120 of the present invention, an ingestible pill including:

an enteric coating; and

a medication-delivery device, which (a) is pliable, (b) is disposed within the enteric coating, having a compressed shape enabled by the pliability, (c) is shaped so as to define (i) one or more medication chambers and (ii) one or more expansible chambers, and (d) includes:
(i) one or more outer surfaces, all of which are pliable;
(ii) hollow medication-delivery needles, which are coupled to at least one of the one or more outer surfaces; and
(iii) a medication, which is contained within the one or more medication chambers,

wherein the medication-delivery device is configured to assume, after the enteric coating dissolves, an expanded shape in which the hollow medication-delivery needles (a) are in fluid communication with the one or more medication chambers and (b) extend away from the medication-delivery device, and

wherein the medication-delivery device is configured such that expansion of the one or more expansible chambers forces the medication from the one or more medication chambers and out of the medication-delivery device through the hollow medication-delivery needles.
Inventive Concept 121. The ingestible pill according to Inventive Concept 120, wherein the medication-delivery device is shaped as a patch.
Inventive Concept 122. The ingestible pill according to Inventive Concept 120, wherein the medication-delivery device is shaped as a circular or elliptical torus.
Inventive Concept 123. The ingestible pill according to any one of Inventive Concepts 120-122, wherein the one or more expansible chambers include one or more inflatable chambers.
Inventive Concept 124. The ingestible pill according to Inventive Concept 123, wherein the one or more inflatable chambers contain a substance that produces gas upon contact with liquid.
Inventive Concept 125. The ingestible pill according to Inventive Concept 124, wherein at least one of the one or more outer surfaces includes biocellulose.

There is yet additionally provided, in accordance with an Inventive Concept 126 of the present invention, an ingestible pill including:

an enteric coating; and

a medication-delivery device, which (a) has a compressed shape when disposed within the enteric coating, (b) is shaped so as to define (i) one or more medication chambers and (ii) one or more inflatable chambers, and (c) includes:
(i) one or more outer surfaces, at least one of which includes biocellulose;
(ii) a substance, which is contained within the one or more inflatable chambers, and which produces gas upon contact with liquid;
(iii) hollow medication-delivery needles, which are coupled to at least one of the one or more outer surfaces; and
(iv) a medication, which is contained within the one or more medication chambers,

wherein the medication-delivery device is configured to assume, after the enteric coating dissolves, an expanded shape in which the hollow medication-delivery needles (a) are in fluid communication with the one or more medication chambers and (b) extend away from the medication-delivery device, and

wherein the medication-delivery device is configured such that inflation of the one or more inflatable chambers forces the medication from the one or more medication chambers and out of the medication-delivery device through the hollow medication-delivery needles.

There is also provided, in accordance with an Inventive Concept 127 of the present invention, an ingestible pill including:

an enteric coating; and

a medication-delivery device, which includes:
(a) a patch, which has a compressed shape when disposed within the enteric coating, and which is shaped so as to define an outer perimeter, and which includes at least first, second, and third layers, which are arranged so as to define:
   (i) one or more medication chambers between the second layer and the third layer, and
   (ii) one or more expansible chambers between the first layer and the second layer, wherein the first layer and the second layer are fixed together at fixation locations, wherein some of the fixation locations are located at or near the outer perimeter and some of the fixation locations are located at least 2 mm from the outer perimeter; and
(b) a medication, which is contained within the one or more medication chambers,

wherein the patch is configured to assume, after the enteric coating dissolves, an expanded shape, and

wherein the third layer is permeable to the medication, such that expansion of the one or more expansible chambers forces the medication from the one or more medication chambers, through the third layer, and out of the medication-delivery device.
Inventive Concept 128. The ingestible pill according to Inventive Concept 127, wherein the patch can inscribe a circle having a diameter of between 2 and 10 cm when the patch assumes the expanded shape and is unconstrained.
Inventive Concept 129. The ingestible pill according to Inventive Concept 127, wherein the patch is configured such that the expanded shape is generally flat when the medication-delivery device is unconstrained.
Inventive Concept 130. The ingestible pill according to Inventive Concept 127, wherein the at least some of the fixation locations are arranged in a plurality of points.
Inventive Concept 131. The ingestible pill according to Inventive Concept 127, wherein the third layer is shaped so as to define one or more pores that provide the permeability.
Inventive Concept 132. The ingestible pill according to Inventive Concept 127, wherein the patch is annular.
Inventive Concept 133. The ingestible pill according to any one of Inventive Concepts 127-132, wherein the medication-delivery device includes a plurality of hollow medication-delivery needles, which are coupled to the third layer, and which, when the patch assumes the expanded shape, are in fluid communication with the one or more medication chambers and extend away from the patch.
Inventive Concept 134. The ingestible pill according to Inventive Concept 133, wherein the ingestible pill is configured such that when the patch assumes the expanded shape upon dissolving of the enteric coating in a small intestine of a subject, the third layer of the patch contacts an intestinal wall, thereby bringing the hollow medication-delivery needles into contact with the intestinal wall.
Inventive Concept 135. The ingestible pill according to Inventive Concept 133, wherein the patch is disposed within the enteric coating, folded so as to define one or more creases, which define respective inner and outer crease sides, and wherein at least 50% of the hollow medication-delivery needles are coupled to the third layer of the patch along the inner crease sides.
Inventive Concept 136. The ingestible pill according to Inventive Concept 135, wherein the patch is disposed within the enteric coating, folded so as to define a plurality of creases.
Inventive Concept 137. The ingestible pill according to Inventive Concept 136, wherein the hollow medication-delivery needles are not coupled to respective inner crease sides of a portion of the creases.
Inventive Concept 138. The ingestible pill according to Inventive Concept 135, wherein between 0% and 10% of the hollow medication-delivery needles are coupled to the patch along the outer crease sides when the patch is disposed within the enteric coating.
Inventive Concept 139. The ingestible pill according to any one of Inventive Concepts 127-132, wherein the at least some of the fixation locations are arranged in a plurality of segments.
Inventive Concept 140. The ingestible pill according to Inventive Concept 139, wherein at least some of the segments are curved when the patch assumes the expanded shape.
Inventive Concept 141. The ingestible pill according to Inventive Concept 140, wherein at least some of the curved segments are arranged equidistantly around a center point of the patch when the patch assumes the expanded shape.
Inventive Concept 142. The ingestible pill according to any one of Inventive Concepts 127-132, wherein the one or more expansible chambers include one or more inflatable chambers.
Inventive Concept 143. The ingestible pill according to Inventive Concept 142, wherein the one or more inflatable chambers contain a substance that produces gas upon contact with liquid.
Inventive Concept 144. The ingestible pill according to any one of Inventive Concepts 127-132, wherein the one or more expansible chambers contain a substance that expands upon contact with liquid.
Inventive Concept 145. The ingestible pill according to Inventive Concept 144, wherein the substance includes a polymer.
Inventive Concept 146. The ingestible pill according to any one of Inventive Concepts 127-132, wherein the first layer is liquid-permeable and substantially not gas-permeable.
Inventive Concept 147. The ingestible pill according to Inventive Concept 146, wherein the first layer includes biocellulose.
There is further provided, in accordance with an Inventive Concept 148 of the present invention, an ingestible pill including:

an enteric coating; and

a medication-delivery device, which includes:
(a) a patch, which has a compressed shape when disposed within the enteric coating, and which is shaped so as to define an outer perimeter, and which includes at least first, second, third, and fourth layers, which are arranged so as to define:
   (i) one or more medication chambers between the third layer and the fourth layer,
   (ii) one or more first expansible chambers between the second and the third layers, and
   (iii) one or more second expansible chambers between the first layer and the second layer, wherein the first layer and the second layer are fixed together at fixation locations, wherein some of the fixation locations are located at or near the outer perimeter and some of the fixation locations are located at least 2 mm from the outer perimeter; and
(b) a medication, which is contained within the one or more medication chambers,

wherein the patch is configured to assume, after the enteric coating dissolves, an expanded shape, and

wherein the fourth layer is permeable to the medication, such that expansion of the one or more second expansible chambers forces the medication from the one or more medication chambers, through the fourth layer, and out of the medication-delivery device.
Inventive Concept 149. The ingestible pill according to Inventive Concept 148, wherein the patch can inscribe a circle having a diameter of between 2 and 10 cm when the patch assumes the expanded shape and is unconstrained.
Inventive Concept 150. The ingestible pill according to Inventive Concept 148, wherein the patch is configured such that the expanded shape is generally flat when the medication-delivery device is unconstrained.
Inventive Concept 151. The ingestible pill according to Inventive Concept 148, wherein at least some of the fixation locations are arranged in a plurality of points.
Inventive Concept 152. The ingestible pill according to Inventive Concept 148, wherein the medication-delivery device is configured such that upon contact with liquid, the one or more second expansible chambers begin to expand before the one or more first expansible chambers begin to expand.
Inventive Concept 153. The ingestible pill according to Inventive Concept 148, wherein the fourth layer is shaped so as to define one or more pores that provide the permeability.
Inventive Concept 154. The ingestible pill according to Inventive Concept 148, wherein the patch is annular.
Inventive Concept 155. The ingestible pill according to any one of Inventive Concepts 148-154, wherein the medication-delivery device includes a plurality of hollow medication-delivery needles, which are coupled to the fourth layer, and, which, when the patch assumes the expanded shape, are in fluid communication with the one or more medication chambers and extend away from the patch.
Inventive Concept 156. The ingestible pill according to Inventive Concept 155, wherein the ingestible pill is configured such that when the patch assumes the expanded shape upon dissolving of the enteric coating in a small intestine of a subject, the fourth layer of the patch contacts an intestinal wall, thereby bringing the hollow medication-delivery needles into contact with the intestinal wall.
Inventive Concept 157. The ingestible pill according to Inventive Concept 155, wherein the patch is disposed within the enteric coating, folded so as to define one or more creases, which define respective inner and outer crease sides, and wherein at least 50% of the hollow medication-delivery needles are coupled to the fourth layer of the patch along the inner crease sides.
Inventive Concept 158. The ingestible pill according to Inventive Concept 157, wherein the patch is disposed within the enteric coating, folded so as to define a plurality of creases.
Inventive Concept 159. The ingestible pill according to Inventive Concept 158, wherein the hollow medication-delivery needles are not coupled to respective inner crease sides of a portion of the creases.
Inventive Concept 160. The ingestible pill according to Inventive Concept 157 wherein between 0% and 10% of the hollow medication-delivery needles are coupled to the patch along the outer crease sides when the patch is disposed within the enteric coating.
Inventive Concept 161. The ingestible pill according to any one of Inventive Concepts 148-154, wherein at least some of the fixation locations are arranged in a plurality of segments.
Inventive Concept 162. The ingestible pill according to Inventive Concept 161, wherein at least some of the segments are curved when the patch assumes the expanded shape.
Inventive Concept 163. The ingestible pill according to Inventive Concept 162, wherein at least some of the curved segments are arranged equidistantly around a center point of the patch when the patch assumes the expanded shape.
Inventive Concept 164. The ingestible pill according to any one of Inventive Concepts 148-154, wherein the one or more second expansible chambers include one or more inflatable chambers.
Inventive Concept 165. The ingestible pill according to Inventive Concept 164, wherein the one or more second inflatable chambers contain a substance that produces gas upon contact with liquid.
Inventive Concept 166. The ingestible pill according to Inventive Concept 165,

wherein the one or more first expansible chambers include one or more first inflatable chambers, and

wherein the second layer is gas-permeable, such that upon inflation of the one or more second inflatable chambers, gas passes from the one or more second inflatable chambers through the gas-permeable second layer to the one or more first inflatable chambers, such that the one or more second inflatable chambers begin to inflate before the one or more first inflatable chambers begin to inflate.
Inventive Concept 167. The ingestible pill according to Inventive Concept 166, wherein the second layer is shaped so as define a plurality of pores that provides the gas-permeability.
Inventive Concept 168. The ingestible pill according to Inventive Concept 166, wherein the second layer is configured to tear upon inflation of the one or more second inflatable chambers, thereby providing the gas-permeability.
Inventive Concept 169. The ingestible pill according to any one of Inventive Concepts 148-154, wherein the one or more second expansible chambers contain a substance that expands upon contact with liquid.
Inventive Concept 170. The ingestible pill according to Inventive Concept 169, wherein the substance includes a polymer.
Inventive Concept 171. The ingestible pill according to any one of Inventive Concepts 148-154, wherein the first layer is liquid-permeable and substantially not gas-permeable.
Inventive Concept 172. The ingestible pill according to Inventive Concept 171, wherein the first layer includes biocellulose.
Inventive Concept 173. The ingestible pill according to any one of Inventive Concepts 148-154, wherein the second layer is liquid-permeable and substantially not gas-permeable.
Inventive Concept 174. The ingestible pill according to Inventive Concept 173, wherein the first layer includes biocellulose.

There is still further provided, in accordance with an Inventive Concept 175 of the present invention, an ingestible pill including:

an enteric coating; and

a medication-delivery device, which has a compressed shape when disposed within the enteric coating, and includes:
a patch having upper and lower surfaces that face in generally opposite directions, wherein the patch includes a non-metal material; and
one or more elastic struts, which are fixed to the patch,

wherein the one or more elastic struts are configured, upon the enteric coating dissolving, to transition the medication-delivery device from the compressed shape to an expanded shape, in which the patch has an outer perimeter.
Inventive Concept 176. The ingestible pill according to Inventive Concept 175, wherein the patch can inscribe a circle having a diameter of between 2 and 10 cm when the patch assumes the expanded shape and is unconstrained.
Inventive Concept 177. The ingestible pill according to Inventive Concept 175, wherein the patch is elliptical or circular.
Inventive Concept 178. The ingestible pill according to Inventive Concept 175, wherein the medication-delivery device further includes a medication.
Inventive Concept 179. The ingestible pill according to Inventive Concept 175, wherein at least 75% of the medication adheres to the upper surface of the patch when the medication-delivery device is disposed in the enteric coating.
Inventive Concept 180. The ingestible pill according to Inventive Concept 175, wherein the patch is configured such that the expanded shape is generally flat when the medication-delivery device is unconstrained.
Inventive Concept 181. The ingestible pill according to Inventive Concept 175, wherein the patch is annular.
Inventive Concept 182. The ingestible pill according to any one of Inventive Concepts 175-181, wherein the one or more elastic struts include metal.
Inventive Concept 183. The ingestible pill according to Inventive Concept 182, wherein the one or more elastic struts include a shape memory alloy.
Inventive Concept 184. The ingestible pill according to Inventive Concept 182, wherein the one or more elastic struts include stainless steel.

There is additionally provided, in accordance with an Inventive Concept 185 of the present invention, a method including:

receiving, by a subject, an ingestible pill, which includes (i) an enteric coating and (ii) a medication-delivery device, which includes (a) a patch having upper and lower surfaces that face in generally opposite directions, and needles, wherein the patch is disposed within the enteric coating, folded so as to define one or more creases, which define respective inner and outer crease sides, wherein at least 50% of the needles are coupled to the patch along the inner crease sides; and

swallowing the ingestible pill by the subject, so that the coating dissolves in a small intestine of the subject and the patch assumes an expanded shape, in which the patch has an outer perimeter.
Inventive Concept 186. The method according to Inventive Concept 185, wherein between 0% and 10% of the needles are coupled to the patch along the outer crease sides when the patch is disposed within the enteric coating.
Inventive Concept 187. The method according to Inventive Concept 185,

wherein the ingestible pill is configured such that when the patch assumes the expanded shape upon dissolving of the enteric coating in the small intestine, the upper surface of the patch contacts an intestinal wall, thereby bringing the needles into contact with the intestinal wall, and

wherein at least 50% of the needles are coupled to the upper surface of the patch along the inner crease sides when the patch is disposed within the enteric coating.
Inventive Concept 188. The method according to Inventive Concept 185, wherein the medication-delivery device further includes a medication, and wherein the needles are configured to deliver the medication.
Inventive Concept 189. The method according to Inventive Concept 185, wherein the needles include a solid medication.
Inventive Concept 190. The method according to Inventive Concept 185,

wherein the patch is disposed within the enteric coating, folded so as to define a plurality of creases, and

wherein the needles are not coupled to respective inner crease sides of a portion of the creases.

There is yet additionally provided, in accordance with an Inventive Concept 191 of the present invention, a method including:

receiving, by a subject, an ingestible pill, which includes (i) an enteric coating and (ii) a medication-delivery device, which includes (a) a patch, which (A) has upper and lower surfaces that face in generally opposite directions, (B) has a compressed shape when disposed within the enteric coating, (C) is shaped so as to define an outer perimeter, and (D) includes at least first and second layers, which are arranged so as to define one or more expansible chambers between the first layer and the second layer, wherein the first layer and the second layer are fixed together at fixation locations, wherein some of the fixation locations are located at or near the outer perimeter and some of the fixation locations are located at least 2 mm from the outer perimeter, and (b) needles, which are coupled to the upper surface at respective needle locations, wherein the patch is configured such that, if the patch were laid generally flat on a flat horizontal surface, at least 80% of the needle locations would be horizontally offset from the fixation locations; and

swallowing the ingestible pill by the subject, so that the coating dissolves in a small intestine of the subject and the patch assumes an expanded shape.

There is also provided, in accordance with an Inventive Concept 192 of the present invention, a method including:

receiving, by a subject, an ingestible pill, which includes (i) an enteric coating and (ii) a medication-delivery device, which (a) is pliable, (b) is disposed within the enteric coating, having a compressed shape enabled by the pliability, (c) is shaped so as to define (A) one or more medication chambers and (B) one or more expansible chambers, and (d) includes (A) one or more outer surfaces, all of which are pliable, (B) hollow medication-delivery needles, which are coupled to at least one of the one or more outer surfaces, and (C) a medication, which is contained within the one or more medication chambers; and

swallowing the ingestible pill by the subject, so that the coating dissolves in a small intestine of the subject and the medication-delivery device assumes an expanded shape in which the hollow medication-delivery needles (a) are in fluid communication with the one or more medication chambers and (b) extend away from the medication-delivery device,

wherein the medication-delivery device is configured such that expansion of the one or more expansible chambers forces the medication from the one or more medication chambers and out of the medication-delivery device through the hollow medication-delivery needles.

There is further provided, in accordance with an Inventive Concept 193 of the present invention, a method including:

receiving, by a subject, an ingestible pill, which includes (i) an enteric coating and (ii) a medication-delivery device, which (a) has a compressed shape when disposed within the enteric coating, (b) is shaped so as to define (A) one or more medication chambers and (B) one or more inflatable chambers, and (c) includes (A) one or more outer surfaces, at least one of which includes biocellulose, (B) a substance, which is contained within the one or more inflatable chambers, and which produces gas upon contact with liquid, (C) hollow medication-delivery needles, which are coupled to at least one of the one or more outer surfaces, and (D) a medication, which is contained within the one or more medication chambers; and

swallowing the ingestible pill by the subject, so that the coating dissolves in a small intestine of the subject and the medication-delivery device assumes an expanded shape an expanded shape in which the hollow medication-delivery needles (a) are in fluid communication with the one or more medication chambers and (b) extend away from the medication-delivery device,

wherein the medication-delivery device is configured such that inflation of the one or more inflatable chambers forces the medication from the one or more medication chambers and out of the medication-delivery device through the hollow medication-delivery needles.

There is still further provided, in accordance with an Inventive Concept 194 of the present invention, a method including:

receiving, by a subject, an ingestible pill, which includes (i) an enteric coating and (ii) a medication-delivery device, which includes (a) a patch, which has a compressed shape when disposed within the enteric coating, and which is shaped so as to define an outer perimeter, and which includes at least first, second, and third layers, which are arranged so as to define (A) one or more medication chambers between the second layer and the third layer, and (B) one or more expansible chambers between the first layer and the second layer, wherein the first layer and the second layer are fixed together at fixation locations, wherein some of the fixation locations are located at or near the outer perimeter and some of the fixation locations are located at least 2 mm from the outer perimeter, and (b) a medication, which is contained within the one or more medication chambers; and

swallowing the ingestible pill by the subject, so that the coating dissolves in a small intestine of the subject and the patch assumes an expanded shape,

wherein the third layer is permeable to the medication, such that expansion of the one or more expansible chambers forces the medication from the one or more medication chambers, through the third layer, and out of the medication-delivery device.

There is additionally provided, in accordance with an Inventive Concept 195 of the present invention, a method including:

receiving, by a subject, an ingestible pill, which includes (i) an enteric coating and (ii) a medication-delivery device, which includes (a) a patch, which has a compressed shape when disposed within the enteric coating, and which is shaped so as to define an outer perimeter, and which includes at least first, second, third, and fourth layers, which are arranged so as to define (A) one or more medication chambers between the third layer and the fourth layer, (B) one or more first expansible chambers between the second and the third layers, and (C) one or more second expansible chambers between the first layer and the second layer, wherein the first layer and the second layer are fixed together at fixation locations, wherein some of the fixation locations are located at or near the outer perimeter and some of the fixation locations are located at least 2 mm from the outer perimeter, and (b) a medication, which is contained within the one or more medication chambers; and

swallowing the ingestible pill by the subject, so that the coating dissolves in a small intestine of the subject and the patch assumes an expanded shape,

wherein the fourth layer is permeable to the medication, such that expansion of the one or more second expansible chambers forces the medication from the one or more medication chambers, through the fourth layer, and out of the medication-delivery device.

There is yet additionally provided, in accordance with an Inventive Concept 196 of the present invention, a method including:

receiving, by a subject, an ingestible pill, which includes (i) an enteric coating and (ii) a medication-delivery device, which has a compressed shape when disposed within the enteric coating, and includes (a) a patch having upper and lower surfaces that face in generally opposite directions, wherein the patch includes a non-metal material, and (b) one or more elastic struts, which are fixed to the patch; and

swallowing the ingestible pill by the subject, so that the coating dissolves in a small intestine of the subject and the one or more elastic struts transition the medication-delivery device from the compressed shape to an expanded shape, in which the patch has an outer perimeter.

The present invention will be more fully understood from the following detailed description of embodiments thereof, taken together with the drawings, in which:

### BRIEF DESCRIPTION OF THE DRAWINGS

FIGS. 1A-B are schematic illustrations of an ingestible pill, for ingestion by a subject, in accordance with an application of the present invention;

FIGS. 2A-F are schematic illustrations of a medication-delivery device of an ingestible pill, in accordance with an application of the present invention;

FIGS. 3A-B are schematic illustrations of a medication-delivery device of another ingestible pill, in accordance with an application of the present invention;

FIGS. 4A-F are schematic illustrations of a medication-delivery device of yet another ingestible pill, in accordance with an application of the present invention;

FIGS. 5A-B are schematic illustrations of a medication-delivery device of still another ingestible pill, in accordance with an application of the present invention;

FIGS. 6A-F are schematic illustrations of a medication-delivery device of another ingestible pill, in accordance with an application of the present invention;

FIGS. 7A-C are schematic illustrations of a medication-delivery device of an ingestible pill, in accordance with an application of the present invention;

FIGS. 8A-B are schematic illustration of a medication-delivery device of an ingestible pill, in accordance with an application of the present invention;

FIGS. 9A-B are schematic illustrations of a medication-delivery device of an ingestible pill, in accordance with an application of the present invention;

FIGS. 10A-B are schematic illustrations of a medication-delivery device of an ingestible pill, in accordance with an application of the present invention;

FIGS. 11A-B are schematic illustrations of a medication-delivery device of an ingestible pill, in accordance with an application of the present invention;

FIGS. 12A-C which are schematic illustrations of configurations of a patch, in accordance with respective applications of the present invention;

FIGS. 13A-D are schematic illustrations of configurations of another patch, in accordance with respective applications of the present invention;

FIG. 14 is a schematic illustration of another ingestible pill, for ingestion by a subject, in accordance with an application of the present invention;

FIG. 15 is a schematic illustration of a medication-delivery device of yet another ingestible pill, for ingestion by a subject, in accordance with an application of the present invention;

FIG. 16 is a schematic illustration of a method of rolling a patch of the medication-delivery devices of FIGS. 14 and 15, in accordance with an application of the present invention;

FIGS. 17A-C are schematic illustrations of three-panel folds known in the art of paper folding; and

FIGS. 18A-D are schematic illustrations of several configurations of a patch of the ingestible pill of FIG. 14, in accordance with respective applications of the present invention.

### DETAILED DESCRIPTION OF EMBODIMENTS

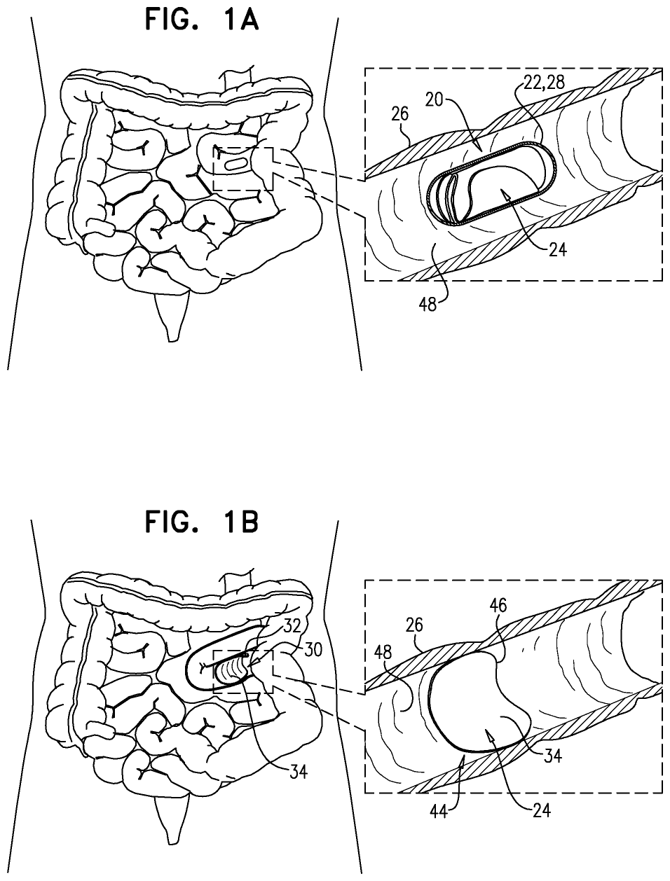

FIGS. 1A-B are schematic illustrations of an ingestible pill 20, for ingestion by a subject, in accordance with an application of the present invention. Ingestible pill 20 comprises an enteric coating 22 and a medication-delivery device 24.

Enteric coating 22 is configured to dissolve in a small intestine 26 (e.g., a duodenum, jejunum, and/or ileum) of the subject. Typically, enteric coating 22 is pH-sensitive, and may be configured to dissolve within 10 minutes (e.g., within 5 minutes) within a range of pH values, which range has a low end of between 6.5 and 8.5 (and, optionally, a high end of between 7.5 and 14, such as between 7.5 and 9.5). For example, enteric coating 22 may comprise gelatin or starch. For some applications, as shown in FIG. 1A, pill 20 comprises a shell 28, which comprises enteric coating 22. Alternatively, for some applications, medication-delivery device 24 is directly coated with enteric coating 22 (configuration not shown). Typically, pill 20 has a length of at least 5 mm, no more than 30 mm, and/or between 5 and 30 mm before the enteric coating dissolves.

Medication-delivery device 24 comprises (a) a patch 30 having an upper (intestinal-wall-contact) surface 32 and a lower (intestinal-lumen-facing) surface 34, which face in generally opposite directions, and (b) needles 40. Medication-delivery device 24 (a) has a compressed shape 42 when disposed within coating 22, as shown in FIG. 1A, and (b) is configured to assume, after enteric coating 22 dissolves, an expanded shape 44, in which patch 30 has an outer perimeter 46. FIG. 1B shows medication-delivery device 24 in expanded shape 44, constrained by a wall 48 of small intestine 26. Typically, patch 30 comprises a non-metal material, such as an elastic material (e.g., polyurethane or silicone), or alternatively a shape memory material such as Nitinol, which, for example, may be set to change into a super-elastic condition after reaching a temperature of 32-34 degrees C. Medication-delivery device 24 expands, such as by stretching, unfolding, and/or unrolling, in response to no longer being constrained by enteric coating 22, and/or in response to contact of medication-delivery device 24 with fluid in small intestine 26.

Typically, each of needles 40 has a length of at least 20 microns (e.g., at least 50 microns), no more than 600 microns (e.g., no more than 300 microns, such as no more than 250 microns), and/or between 20 and 600 microns, e.g., between 20 and 600 microns, e.g., between 20 and 300 microns, such as between 50 and 250 microns. Typically, a greatest dimension of needles 40, measured perpendicular to a long axis, is between 40 and 300 microns. For some applications, needles 40 are conical or pyramidal, and the greatest dimension is thus at the bases of the cones or pyramids of the needles.

For some applications, medication-delivery device 24 is biodegradable in small intestine 26. For some applications, patch 30 comprises an elastomer, such as polyethylene or silicone.

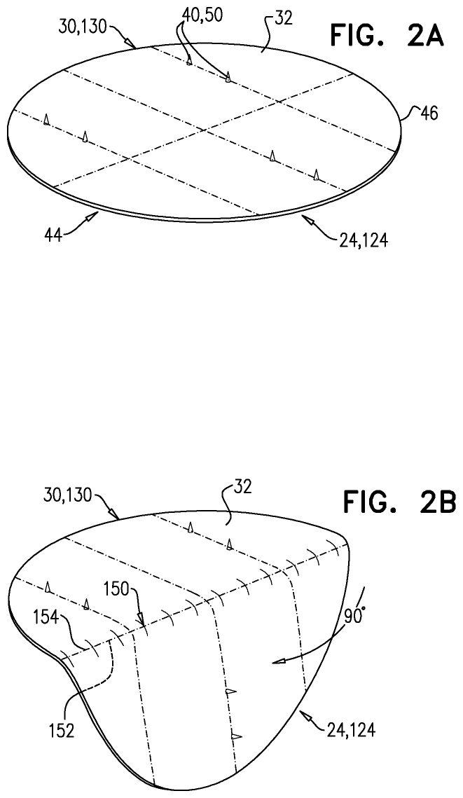

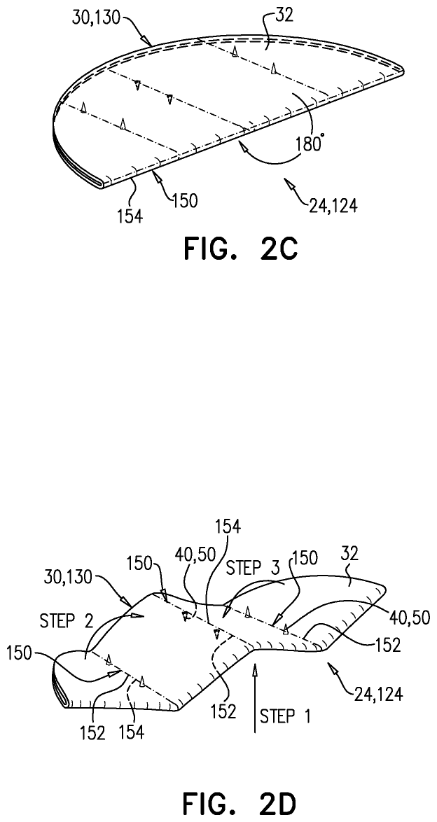

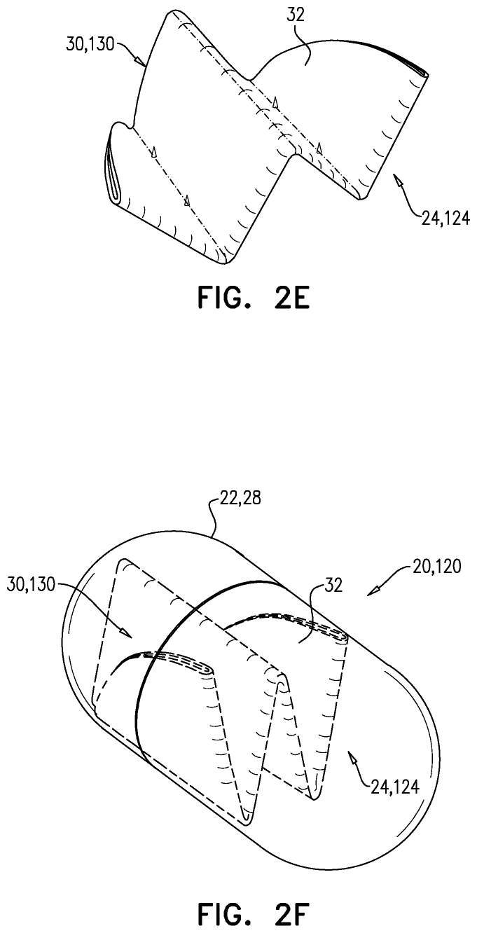

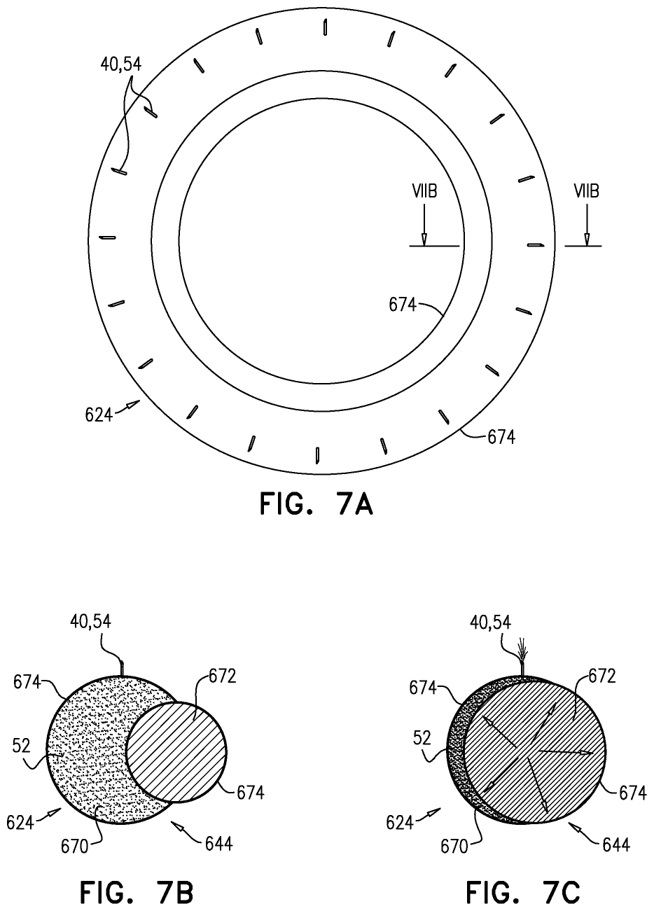

For some applications, such as shown in FIGS. 1A-B and described hereinbelow with reference to FIGS. 2A-F, 3A-B, 4A-F, 5A-B, and 6A-F, needles 40 are medication-needles 50, which comprise a solid medication (for example, the solid medication may be integrated in a glucose structure). For other applications, such as described hereinbelow with reference to FIGS. 7A-C, 8A-B, 9A-B, and 10A-B, medication-delivery device 24 further comprises a medication 52 (e.g., a liquid medication), and needles 40 are medication-delivery needles 54, which are configured to deliver medication 52. For these applications, medication-delivery needles 54 are typically hollow and are configured to deliver medication 52.

Typically, patch 30 is configured such that expanded shape 44 is generally flat when medication-delivery device 24 is unconstrained, such as shown in FIGS. 2A, 3A, 4A, 5A, and 6A, and assumes a curved shape when constrained by wall 48 of small intestine 26, such as shown in FIG. 1B. Ingestible pill 20 is configured such that when patch 30 assumes expanded shape 44 upon dissolving of enteric coating 22 in small intestine 26, upper surface 32 of patch 30 contacts intestinal wall 48, thereby bringing needles 40 into contact with intestinal wall 48. Typically, but not necessarily, needles 40 penetrate intestinal wall 48, typically the villi thereof, in order to enhance uptake of the medication. In addition, needles 40 may also serve as temporary anchors that hold patch 30 in place during medication delivery. Medication-delivery device 24 typically remains axially stationary in the small intestine long enough for needles 40 to slowly push into tissue of intestinal wall 48, such as because of peristalsis, and to inject medication, for applications in which the needles are hollow, or to dissolve, for applications in which the needles comprise medication. For some applications in which medication-delivery device 24 expands (e.g., inflates), the expansion itself does not generally push the needles into the intestinal wall. Following delivery of the medication, medication-delivery device 24 is passed from the body.

Once expanded, upper surface 32 of patch 30 establishes good (complete or nearly complete) contact with intestinal wall 48, as shown in FIG. 1B. The shape and dimensions of medication-delivery device 24 may contribute to this good contact by preventing other, lower-contact-level orientations of the patch in the lumen of small intestine 26. The location of patch 30 is generally unaffected by peristalsis, and does not block food passing through the intestine.

For some applications, medication-delivery device 24 further comprises a mucoadhesive that at least partially coats upper surface 32. For example, the mucoadhesive may be sprayed or printed on upper surface 32 using techniques known in the art. The mucoadhesive transiently helps adhere upper surface 32 in position against intestinal wall 48 during delivery of the medication through intestinal wall 48. For example, the mucoadhesive may include an adhesive agent described in U.S. Pat. No. 6,235,313 to Mathiowitz et al., or in an article by Tao et al., Tao et al., entitled, “Gastrointestinal patch systems for oral drug delivery,” Drug Discovery Today, Vol. 10(13), July 2005, both of which references are incorporated herein by reference.

Reference is made to FIGS. 2A-F, which are schematic illustrations of a medication-delivery device 124 of an ingestible pill 120, in accordance with an application of the present invention. FIG. 2A shows medication-delivery device 124 when unconstrained in expanded shape 44, FIGS. 2B-E show medication-delivery device 124 in various stages of folding, and FIG. 2F shows medication-delivery device 124 folded within enteric coating 22. Medication-delivery device 124 is one configuration of medication-delivery device 24, described hereinabove with reference to FIGS. 1A-B, and may implement any of the features described with reference to FIGS. 1A-B. Medication-delivery device 124 comprises a patch 130, which is one configuration of patch 30, described hereinabove with reference to FIGS. 1A-B, and may implement any of the features described with reference to FIGS. 1A-B. In general, the folding pattern and disposition of patch 130 within enteric coating 22 determines which surface of the patch comes in contact with intestinal wall 48 upon dissolving of enteric coating 22.

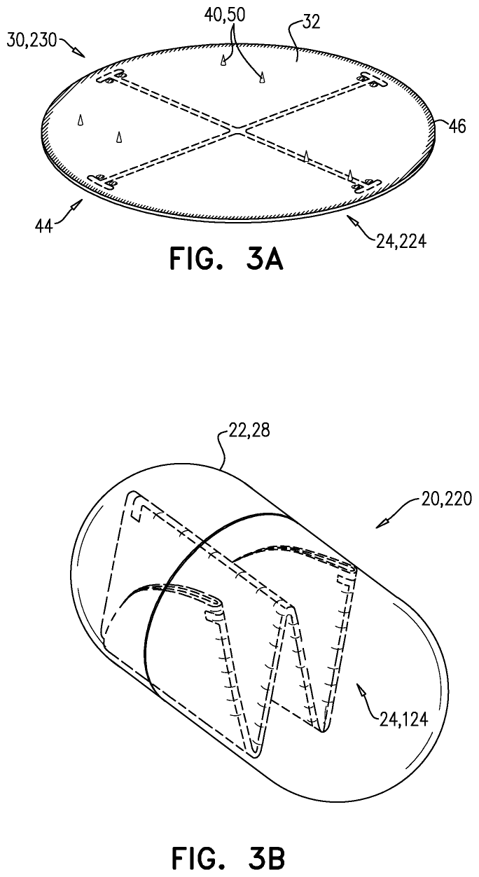

Reference is made to FIGS. 3A-B, which are schematic illustrations of a medication-delivery device 224 of an ingestible pill 220, in accordance with an application of the present invention. FIG. 3A shows medication-delivery device 224 when unconstrained in expanded shape 44, and FIG. 3B shows medication-delivery device 224 folded within enteric coating 22. Medication-delivery device 224 is one configuration of medication-delivery device 24, described hereinabove with reference to FIGS. 1A-B, and may implement any of the features described with reference to FIGS. 1A-B. Other than as described below, medication-delivery device 224 is similar to medication-delivery device 124, and may implement any of the features thereof. Medication-delivery device 224 comprises a patch 230, which is one configuration of patch 30, described hereinabove with reference to FIGS. 1A-B, and may implement any of the features described with reference to FIGS. 1A-B.

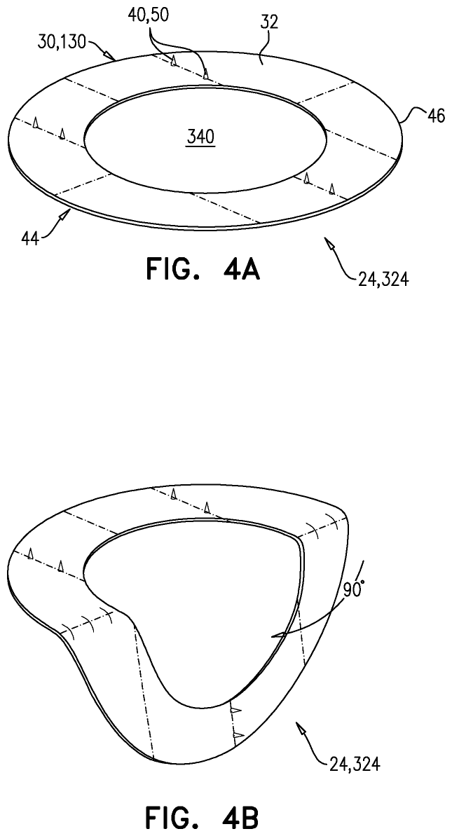

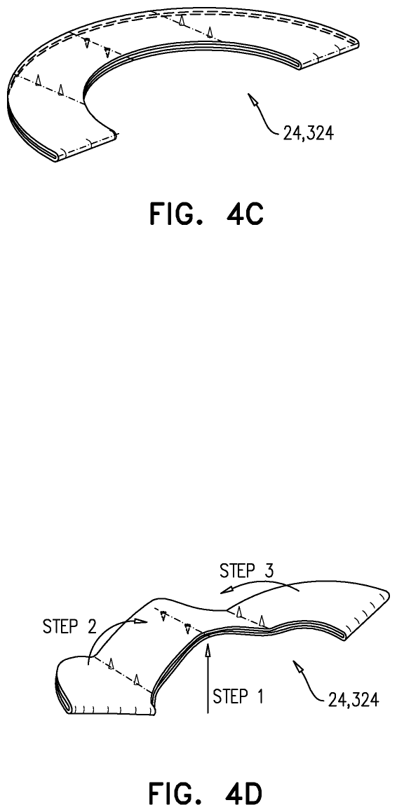

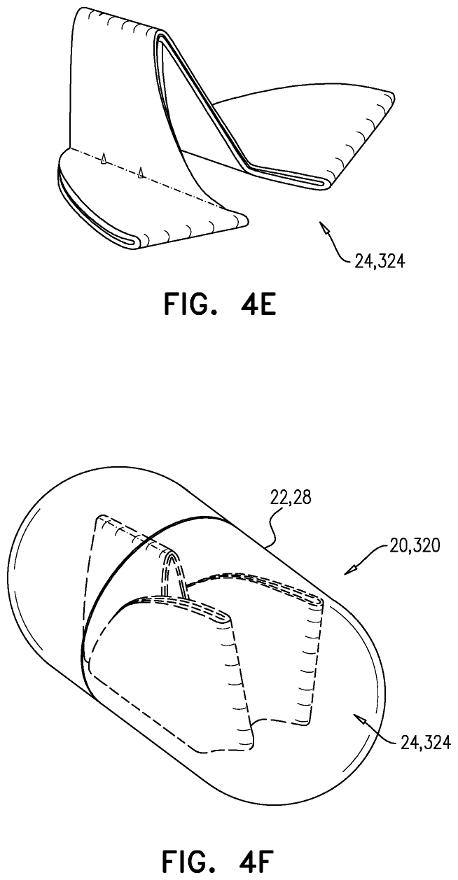

Reference is made to FIGS. 4A-F, which are schematic illustrations of a medication-delivery device 324 of an ingestible pill 320, in accordance with an application of the present invention. FIG. 4A shows medication-delivery device 324 when unconstrained in expanded shape 44, FIGS. 4B-E show medication-delivery device 124 in various stages of folding, and FIG. 4F shows medication-delivery device 324 folded within enteric coating 22. Medication-delivery device 324 is one configuration of medication-delivery device 24, described hereinabove with reference to FIGS. 1A-B, and may implement any of the features described with reference to FIGS. 1A-B. Other than as described below, medication-delivery device 324 is similar to medication-delivery device 124, and may implement any of the features thereof. Medication-delivery device 324 comprises a patch 330, which is one configuration of patch 30, described hereinabove with reference to FIGS. 1A-B, and may implement any of the features described with reference to FIGS. 1A-B.

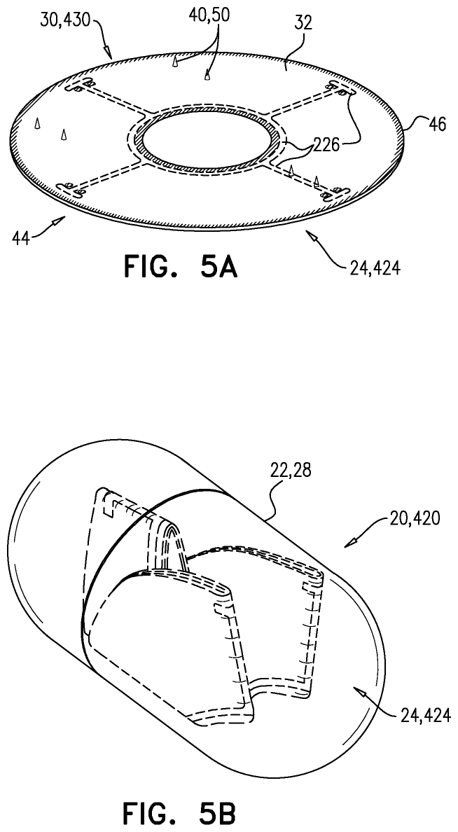

Reference is made to FIGS. 5A-B, which are schematic illustrations of a medication-delivery device 424 of an ingestible pill 420, in accordance with an application of the present invention. FIG. 5A shows medication-delivery device 424 when unconstrained in expanded shape 44, and FIG. 5B shows medication-delivery device 424 folded within enteric coating 22. Medication-delivery device 424 is one configuration of medication-delivery device 24, described hereinabove with reference to FIGS. 1A-B, and may implement any of the features described with reference to FIGS. 1A-B. Other than as described below, medication-delivery device 424 is similar to medication-delivery device 324, and may implement any of the features thereof. Medication-delivery device 424 comprises a patch 130, which is one configuration of patch 430, described hereinabove with reference to FIGS. 1A-B, and may implement any of the features described with reference to FIGS. 1A-B.

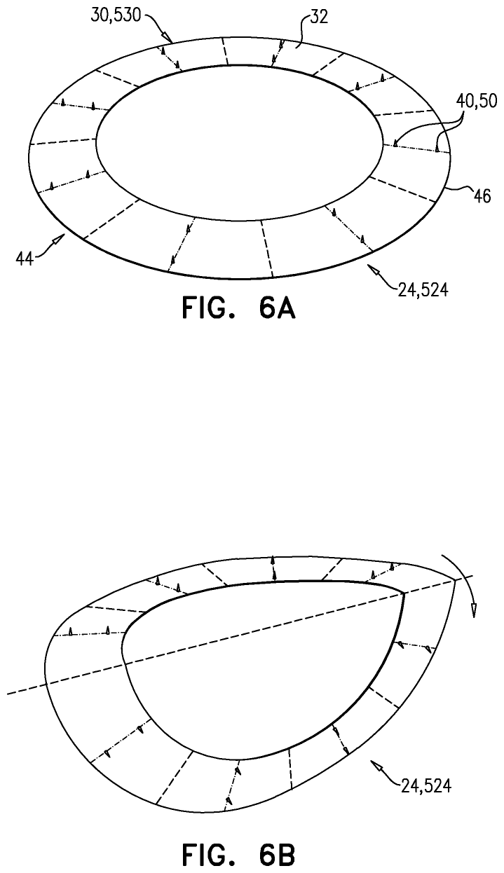

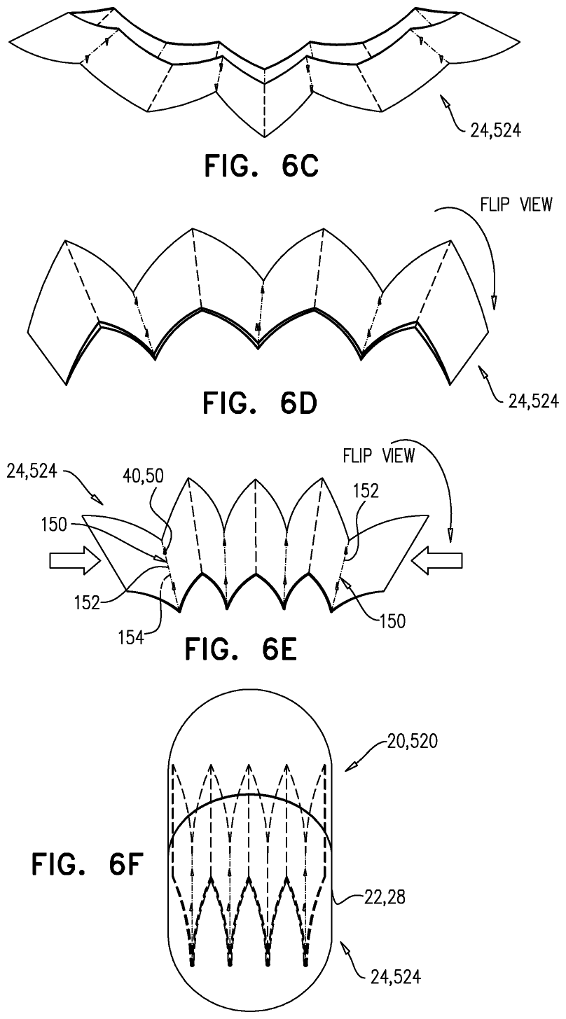

Reference is made to FIGS. 6A-F, which are schematic illustrations of a medication-delivery device 524 of an ingestible pill 520, in accordance with an application of the present invention. FIG. 6A shows medication-delivery device 524 when unconstrained in expanded shape 44, FIGS. 6B-E show medication-delivery device 124 in various stages of folding, and FIG. 6F shows medication-delivery device 524 folded within enteric coating 22. Medication-delivery device 524 is one configuration of medication-delivery device 24, described hereinabove with reference to FIGS. 1A-B, and may implement any of the features described with reference to FIGS. 1A-B. Other than as described below, medication-delivery device 524 is similar to medication-delivery device 324, and may implement any of the features thereof. Medication-delivery device 524 comprises a patch 530, which is one configuration of patch 30, described hereinabove with reference to FIGS. 1A-B, and may implement any of the features described with reference to FIGS. 1A-B.

Reference is made to FIGS. 2A-F, 3A-B, 4A-F, 5A-B, and 6A-F. As shown in FIGS. 2F, 3B, 4F, 5B, and 6F, patch 30 is disposed within enteric coating 22, folded so as to define one or more creases 150 (e.g., a plurality of creases 150), which define respective inner crease sides 152 and outer crease sides 154. The formation of these creases is illustrated in FIGS. 2A-E, 3A, 4A-E, 5A, and 6A-E, respectively. As used in the present application, including in the claims, a “crease” is a sharp, well-defined fold. For some applications, at least 50% (such as at least 75%, e.g., at least 90%, e.g., 100%) of needles 40 are coupled (e.g., adhered) to patch 30 along inner crease sides 152. Coupling needles 40 to patch along inner crease sides 152 helps protect the needles from being broken during folding of the patch and stabilizes the needles on either side of the crease, and helps avoiding deflecting the needles from the plane of the surface when the patch is folded.

For some applications, patch 30 is disposed within enteric coating 22, folded first in half (as can perhaps best be seen in FIGS. 2B-C, 4B-C, and 6B), and then accordion-folded, as can be seen in FIGS. 2D-F, 3B, 4D-F, 5B, and 6C-F.

Typically, patch 30 is disposed within enteric coating 22, folded such that when patch 30 assumes expanded shape 44 upon dissolving of enteric coating 22 in small intestine 26, upper surface 32 of patch 30 contacts intestinal wall 48, thereby bringing needles 40 into contact with intestinal wall 48. In other words, the folding of patch 30 within enteric coating 22 typically determines which surface of patch 30 comes in contact with intestinal wall 48.

Typically, needles 40 are not coupled to respective inner crease sides 152 of a portion of creases 150. Also typically, needles 40 are not coupled to respective inner crease sides 152 of two or more of creases 150.

For some applications, at least 50%, such as at least 80%, e.g., 100%, of needles 40 are coupled to upper surface 32 of patch 30 when patch 30 is disposed within enteric coating 22. Alternatively or additionally, for some applications, at least 50% of needles 40 are coupled to upper surface 32 of patch 30 along inner crease sides 152 when patch 30 is disposed within enteric coating 22.

Typically, between 0% (i.e., none) and 10% of needles 40 are coupled to patch 30 along outer crease sides 154 when patch 30 is disposed within enteric coating 22.

Reference is made to FIGS. 2A, 3A, 4A, 5A, and 6A, which show patch 30 generally flat in expanded shape 44 when medication-delivery device 24 is unconstrained. For some applications, patch 30 can inscribe a circle having a diameter of at least 2 cm, less than 10 cm (e.g., less than 7 cm, such as less than 5 cm), and/or between 2 and 10 cm, e.g., between 2 and 7 cm, such as between 2 and 5 cm), when patch 30 assumes expanded shape 44 and is unconstrained. This diameter may allow patch 30 to circumscribe at least 180 degrees of the intestinal lumen, but typically not more than 360 degrees. (As is known in the art, an inscribed circle is the largest possible circle that can be drawn on the inside of a plane figure. It is noted that the circle is a geometric shape used to describe patch 30, and is not an element of the invention.) Alternatively or additionally, for applications in which patch 30 is circular, the diameter of patch 30 is typically greater than π (pi) times the radius of small intestine 26, and/or greater than 60 mm or between 65 mm and 150 mm, such that patch 30 extends around at least 180 degrees of a circumference of small intestine 26 when expanded in small intestine 26.

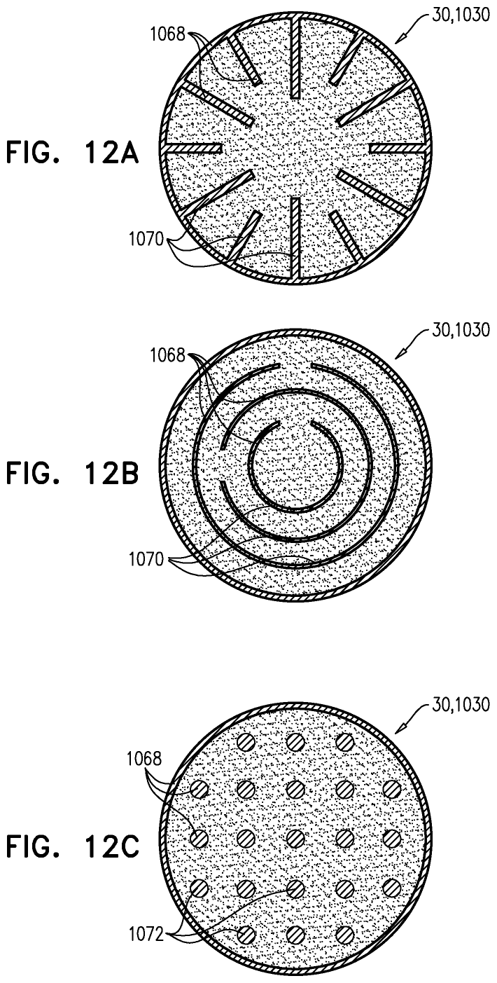

Reference is made to FIGS. 4A-F, 5A-B, 6A-F, and 13A-D. In the configurations shown in these figures, patch 30 is annular, i.e., is shaped as a ring defining an opening 340 therethrough when unconstrained in expanded shape 44, as shown in FIGS. 4A, 5A, 6A, and 13A-D. Opening 340 may allow for compression of medication-delivery device 24 when in compressed shape 42 when disposed within enteric coating 22, while still providing good contact with intestinal wall 48 when in expanded shape 44.

Reference is made to FIGS. 3A-B and 5A-B. In the configurations shown in these figures, patch 30 comprises a non-metal material, and medication-delivery device 224, 424 further comprises elastic struts 226, which are fixed to patch 30. Elastic struts 226 are configured, upon enteric coating 22 dissolving, to transition medication-delivery device 224, 424 from the compressed shape to expanded shape 44.

For some applications, elastic struts 226 comprise metal, such as a shape memory alloy, such as Nitinol. The low stress of the shape memory alloy when the medication-delivery device has compressed shape 42 when disposed within coating 22 reduces the likelihood of the medication-delivery device forgetting its memorized shape. Alternatively, for some applications, struts 226 comprise stainless steel.

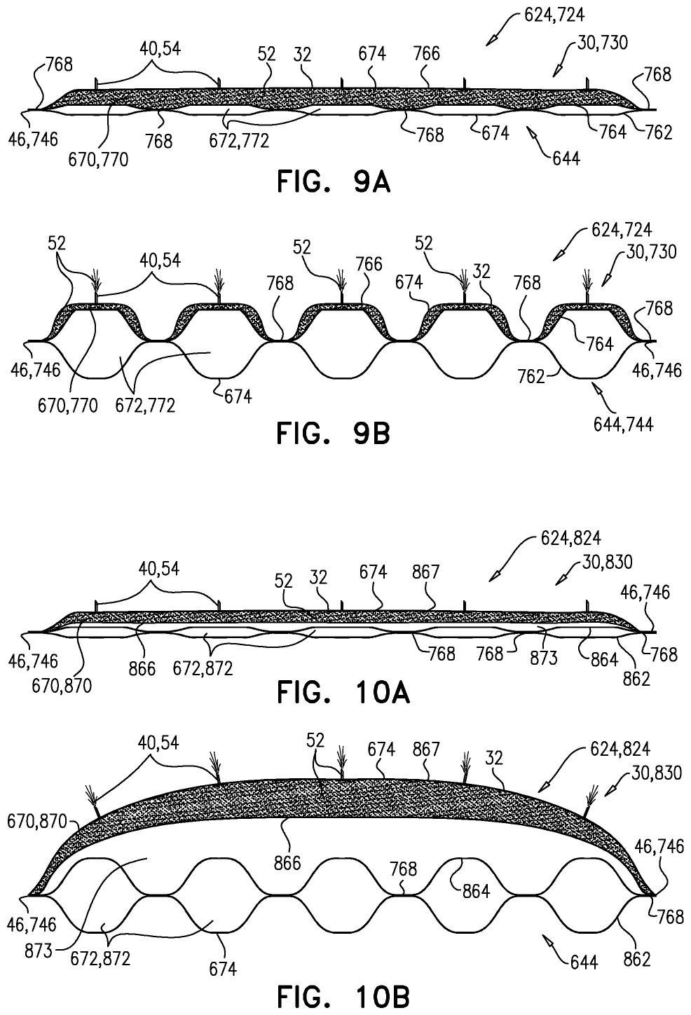

Optionally, patch 30 comprises a plurality of layers, which are fixed together (for example, fused (e.g., welded) or glued together) at one or more fixation locations 768, such as shown, for example, in FIGS. 9A-B and 10A-B. All of the layers described herein may comprise one or more pieces of material, and multiple layers may optionally be formed from a single folded piece of material. The layers described herein comprise a broad, thin, pliable material, e.g., a membrane.

Reference is made to FIGS. 7A-13D, which are schematic illustrations of several configurations of a medication-delivery device 624, in accordance with respective applications of the present invention. Medication-delivery device 624 may implement any of the features of medication-delivery device 24, described hereinabove with reference to FIGS. 1A-6F. Medication-delivery device is a component of ingestible pill 20 (which comprises enteric coating 22), described hereinabove with reference to FIGS. 1A-6F.

FIGS. 7A-C show medication-delivery device 624 in an expanded shape 644 after enteric coating 22 dissolves. FIG. 7B shows medication-delivery device 624 before expansion of one or more expansible chambers 672, as described below. FIGS. 7A and 7C show medication-delivery device 624 upon expansion of the one or more expansible chambers 672, as described below.

In the configurations described with reference to FIGS. 7A-13D, medication-delivery device 624 is pliable, i.e., easily bent and flexible, as opposed to stiff or rigid. Medication-delivery device 624 is disposed within enteric coating 22, having a compressed shape enabled by the pliability (similar to the configuration shown in FIG. 1A). The pliability also typically helps medication-delivery device 624 conform with intestinal wall 48 when in expanded shape 644.

Medication-delivery device 624 is shaped so as to define one or more medication chambers 670 and one or more expansible chambers 672. Medication-delivery device 624 comprises:
one or more outer surfaces 674, all of which are pliable;
hollow medication-delivery needles 54, which are coupled to at least one of the one or more outer surfaces 674; and
medication 52, which is contained within the one or more medication chambers 670.

Medication-delivery device 624 is configured to assume, after enteric coating 22 dissolves, expanded shape 644 in which hollow medication-delivery needles 54 (a) are in fluid communication with the one or more medication chambers 670 and (b) extend away from medication-delivery device 624. Medication-delivery device 624 is configured such that expansion of the one or more expansible chambers 672 forces medication 52 from the one or more medication chambers 670 and out of medication-delivery device 624 through hollow medication-delivery needles 54. For example, medication 52 may comprise a liquid, or a solid that dissolves upon exposure to bodily fluids.

For some applications, the one or more expansible chambers 672 comprise one or more inflatable chambers. For some applications, the one or more inflatable chambers contain a substance that produces gas upon contact with liquid, such as described hereinbelow with reference to FIGS. 9A-B.

For some applications, at least one of the one or more outer surfaces 674 comprises biocellulose (also known in the art as microbial cellulose and bacterial cellulose), which is liquid-permeable and substantially not gas-permeable.

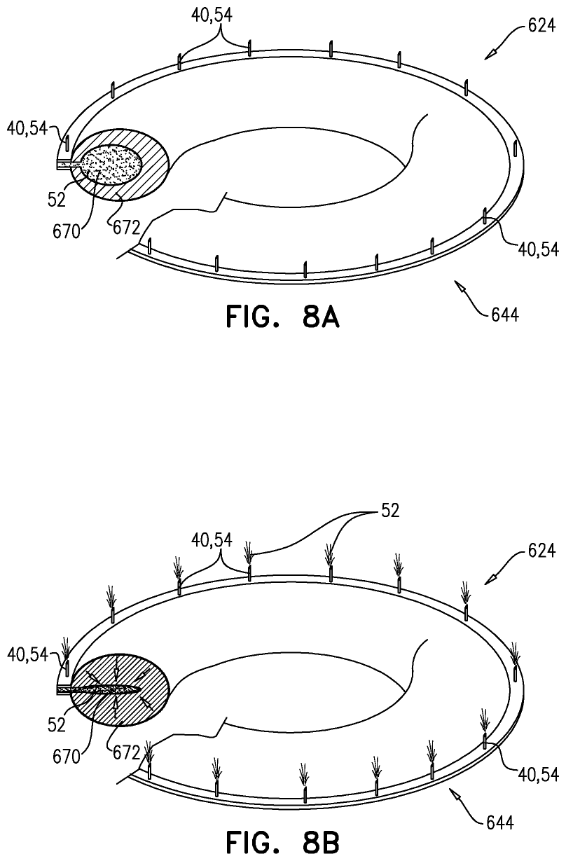

Reference is made to FIGS. 7A-8B. For some applications, medication-delivery device 624 is shaped as a circular or elliptical torus. The toroidal shape provides the above-mentioned structure.

Reference is made to FIGS. 9A-13D. For some applications, medication-delivery device 624 is shaped as a patch 30.

Reference is made to FIGS. 9A-B, which are schematic illustrations of a medication-delivery device 724, in accordance with an application of the present invention. FIG. 9A shows medication-delivery device 724 after enteric coating 22 has dissolved. FIG. 9B shows medication-delivery device 724 after expansion of expansible chambers 672, 772, as described hereinbelow. Medication-delivery device 724 is one implementation of medication-delivery device 624, described hereinabove, and may implement any of the features thereof. In addition, medication-delivery device 724 may implement any of the features of the medication-delivery devices described herein with reference to FIGS. 1A-B, 2A-F, 3A-B, 4A-F, 5A-B, 6A-F, 11A-B, 12A-C, and/or 13A-D.

Medication-delivery device 724 is a component of an ingestible pill comprising enteric coating 22. Medication-delivery device 724 comprises a patch 730, which has a compressed shape when disposed within enteric coating 22, and which is shaped so as to define an outer perimeter 46, 746, and which comprises at least a first layer 762, a second layer 764, and a third layer 766, which are arranged so as to define:
one or more medication chambers 670, 770 between second layer 764 and third layer 766, and
one or more expansible chambers 672, 772 between first layer 762 and second layer 764.

First layer 762 and second layer 764 are fixed together at fixation locations 768, for example, fused (e.g., welded) or glued together. Some of fixation locations 768 are located at or near outer perimeter 760 and some of fixation locations 768 are located at least 2 mm from outer perimeter 760. Fixation locations 768 generally at least partially divide an interior of the one or more expansible chambers 672, 772 into sub-chambers, which help maintain patch 730 fairly flat even upon inflation.

Medication-delivery device 724 further comprises medication 52, which is contained within the one or more medication chambers 670, 770.

Patch 730 is configured to assume, after enteric coating 22 dissolves, an expanded shape 644, 744, such as shown in FIG. 9B.

Third layer 766 is permeable to medication 52, such that expansion of the one or more expansible chambers 672, 772 forces medication 52 from the one or more medication chambers 670, 770, through third layer 766, and out of medication-delivery device 724. Third layer 766 thus serves as upper surface 32 of patch 730 that contact intestinal wall 48, as described hereinabove.

For some applications, the one or more expansible chambers 672, 772 comprise one or more inflatable chambers. Typically, the one or more inflatable chambers contain a substance that produces gas upon contact with liquid. For example, the substance may comprise sodium bicarbonate and/or citric acid (e.g., 60-70% sodium bicarbonate and 30-40% citric acid). For some applications, first layer 762 is liquid-permeable and substantially not gas-permeable, in order to allow bodily fluids to pass into the one or more expansible chambers 672, 772 and contact the substance that produces the gas. For example, first layer 762 may comprise biocellulose. Optionally, the entire patch 730, except the upper surface that faces the intestinal wall, comprises biocellulose.

For other applications, the one or more expansible chambers 672, 772 contain a substance that expands upon contact with liquid. For example, the substance may comprise a polymer, such as a hydrogel.

For some applications, patch 730 can inscribe a circle having a diameter of at least 2 cm, less than 10 cm (e.g., less than 7 cm, such as less than 5 cm), and/or between 2 and 10 cm, e.g., between 2 and 7 cm, such as between 2 and 5 cm), when patch 30 assumes expanded shape 644, 744. Typically, patch 730 is configured such that expanded shape 644, 744 is generally flat when medication-delivery device 724 is unconstrained and becomes curved by intestinal wall 48.

For some applications, medication-delivery device 724 comprises a plurality of hollow medication-delivery needles 40, 54, which are coupled to third layer 766 in fluid communication with the one or more medication chambers 670 when medication-delivery device 724 is in expanded shape 644, 744, and extend away from patch 730 when patch 730 assumes expanded shape 644, 744. Typically, the ingestible pill is configured such that when patch 730 assumes expanded shape 644, 744 upon dissolving of enteric coating 22 in small intestine 26, third layer 766 of patch 30 contacts intestinal wall 48, thereby bringing hollow medication-delivery needles 54 into contact with intestinal wall 48.

For other applications, third layer 766 is shaped so as to define one or more pores that provide the permeability.

Reference is made to FIGS. 10A-B, which are schematic illustrations of a medication-delivery device 824, in accordance with an application of the present invention. FIG. 10A shows medication-delivery device 824 after enteric coating 22 has dissolved. FIG. 10B shows medication-delivery device 824 after expansion of first expansible chambers 873 and second expansible chambers 672, 872, as described hereinbelow. Medication-delivery device 824 is one implementation of medication-delivery device 624, described hereinabove, and may implement any of the features thereof. In addition, medication-delivery device 824 may implement any of the features of the medication-delivery devices described herein with reference to FIGS. 1A-B, 2A-F, 3A-B, 4A-F, 5A-B, 6A-F, 9A-B, 11A-B, 12A-C, and/or 13A-D.

Other than as described below, medication-delivery device 824 is identical to medication-delivery device 724, described hereinabove with reference to FIGS. 9A-B.

A patch 830 of medication-delivery device 824 comprises at least a first layer 862, a second layer 864, a third layer 866, and a fourth layer 867, which are arranged so as to define:
one or more medication chambers 670, 870 between third layer 866 and fourth layer 867,
one or more first expansible chambers 873 between second layer 864 and third layer 866, and
one or more second expansible chambers 672, 872 between first layer 862 and second layer 864.

First layer 862 and second layer 864 are fixed together at fixation locations 768. Some of the fixation locations 868 are located at or near outer perimeter 46, 746 and some of fixation locations 768 are located at least 2 mm from outer perimeter 46, 746.

Fourth layer 867 is permeable to medication 52, such that expansion of the one or more second expansible chambers 672, 872 forces medication 52 from the one or more medication chambers 670, 870, through fourth layer 867, and out of medication-delivery device 824. Fourth layer 867 thus serves as upper surface 32 of patch 830 that contact intestinal wall 48, as described hereinabove.

For some applications, medication-delivery device 824 is configured such that upon contact with liquid, the one or more second expansible chambers 672, 872 begin to expand before the one or more first expansible chambers 873 begin to expand. For example, the liquid may have separate access to the one or more second expansible chambers 672, 872 and the one or more first expansible chambers 873, and a physical property of the one of or more of the layers is configured to control timing. For some applications, the one or more first expansible chambers 873 comprise one or more first inflatable chambers, and second layer 864 is gas-permeable, such that upon inflation of the one or more second inflatable chambers, gas passes from the one or more second inflatable chambers through the gas-permeable second layer 864 to the one or more first inflatable chambers, such that the one or more second inflatable chambers begin to inflate before the one or more first inflatable chambers begin to inflate, which in turn force the medication 52 through fourth layer 867. For some applications, second layer 864 is shaped so as define a plurality of pores (i.e., small holes) that provides the gas-permeability. Alternatively or additionally, for some applications, second layer 864 is configured to tear upon inflation of the one or more second inflatable chambers, thereby providing the gas-permeability.

For some applications, first layer 862 is liquid-permeable and substantially not gas-permeable, such as described above with reference to FIGS. 9A-B. For example, first layer 862 may comprise biocellulose.

For some applications, second layer 864 is liquid-permeable and substantially not gas-permeable. For some applications, first layer 862 comprises biocellulose.

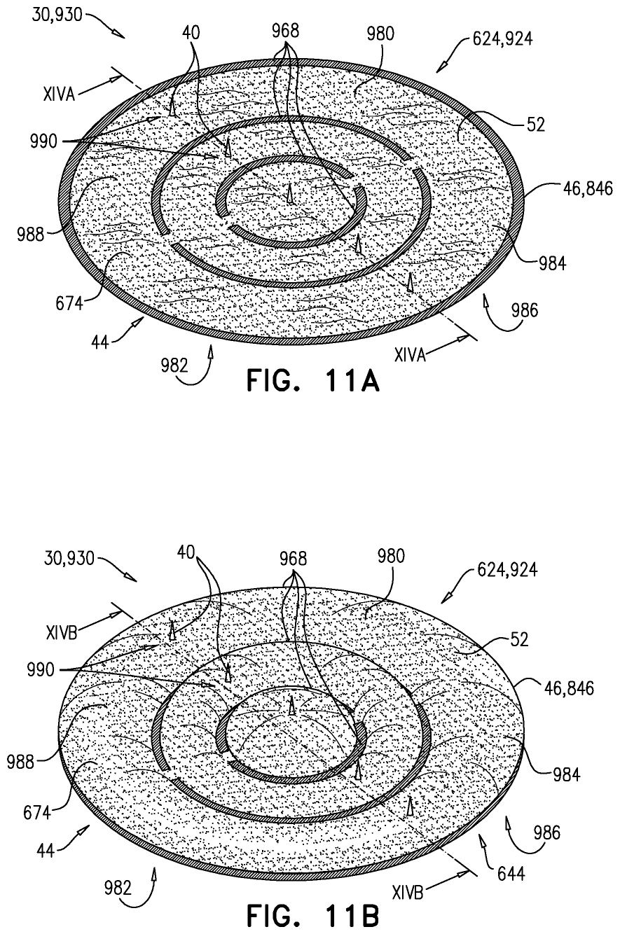

Reference is again made to FIGS. 9A-B and 10A-B, and is additionally made to FIGS. 11A-B, which are schematic illustrations of a medication-delivery device 924, in accordance with an application of the present invention. Medication-delivery device 924 is a component of an ingestible pill comprising enteric coating 22. Medication-delivery device 924 is one implementation of medication-delivery device 624, described hereinabove, and may implement any of the features thereof. In addition, medication-delivery device 924 may implement any of the features of the medication-delivery devices described herein with reference to FIGS. 1A-B, 2A-F, 3A-B, 4A-F, 5A-B, 6A-F, 10A-B, 12A-C, and/or 13A-D.

FIG. 11A shows medication-delivery device 924 after enteric coating 22 has dissolved. FIG. 11B shows medication-delivery device 924 after expansion of expansible chambers 988 thereof. For example, medication-delivery device 924 may be shaped so as to define expansible chambers 772, such as described hereinabove with reference to FIGS. 9A-B, or first expansible chambers 873 and second expansible chambers 872, such as described hereinabove with reference to FIGS. 10A-B. The expansible chambers may implement any of the features of the expansible chambers described herein.

Medication-delivery device 924 comprises (a) a patch 930, which has a compressed shape when disposed within enteric coating 22, and which is shaped so as to define an outer perimeter 46, 846, and (b) needles 40.

Patch 930 has an upper surface 980 and a lower surface 982 that face in generally opposite directions. Patch 930 (iv) comprises at least a first layer 984 and a second layer 986, which are arranged so as to define one or more expansible chambers 988 between first layer 984 and second layer 986. First layer 984 and second layer 986 are fixed together at fixation locations 968. Typically, some of fixation locations 968 are located at or near outer perimeter 46, 846 and some of fixation locations 968 are located at least 2 mm from outer perimeter 46, 846. Needles 40 are coupled to upper surface 980 at respective needle locations 990.

Patch 930 is configured such that, if patch 930 were laid generally flat on a flat horizontal surface, at least 80%, e.g., at least 90%, such as all, of needle locations 990 would be horizontally offset from fixation locations 968. As used in the present application, including in the claims, “horizontally” means in a direction along the plane defined by the flat horizontal surface. It is noted that flat horizontal surface is not an element of the invention, but instead is a geometric shape used to define certain structural features of the invention. The invention does not require actually placing the patch laid flat on the flat horizontal surface.

For some applications, patch 930 is configured such that, if patch 930 were laid generally flat on the flat horizontal surface, needle locations 990 would be located on average at least 1 mm (e.g., at least 2 mm, such as at least 3 mm, e.g., at least 5 mm) horizontally from respective nearest fixation locations 968. For some applications, patch 930 is configured such that, if patch 930 were laid generally flat on the flat horizontal surface, each of needle locations 990 would be located at least 1 mm (e.g., at least 2 mm, such as at least 3 mm, e.g., at least 5 mm) horizontally from a nearest one of fixation locations 968.

For some applications, patch 930 is configured such that, if patch 930 were laid generally flat on the flat horizontal surface, each of needle locations 990 would be located approximately horizontally centered between a nearest two of fixation locations 968.

The arrangements of needle locations 990 with respect to fixation locations 968 described above result in needles 40 being generally located at or near local peaks of upper surface 980 between valleys defined by fixation locations 968. These relatively high locations may help maximize the puncturing of needles 40 through villi of intestinal wall 48 with as short as possible needles, and with minimal risk of blocking the needles with the expandable chamber before all of the drug is expelled (i.e., with minimum residual drug).

Needles 40 may have any of the features described herein. For example, the needles may comprise a solid medication, or may be configured to deliver a medication contained in medication-delivery device 924, such as in one or more medication chambers, such as described herein.

For some applications, patch 930 is disposed within enteric coating 22, folded so as to define one or more creases (e.g., a plurality of creases), which define respective inner and outer crease sides, and wherein at least 50% of the needles are coupled to upper surface 980 of patch 930 along the inner crease sides, such as described hereinabove with reference to FIGS. 2A-F, 3A-B, 4A-F, 5A-B, and 6A-F.

For some applications, patch 930 can inscribe a circle having a diameter of at least 2 cm, less than 10 cm (e.g., less than 7 cm, such as less than 5 cm), and/or between 2 and 10 cm, e.g., between 2 and 7 cm, such as between 2 and 5 cm, when patch 930 assumes the expanded shape and is unconstrained.

Reference is now made to FIGS. 12A-C and 13A-D, which are schematic illustrations of configurations of a patch 30, 1030, in accordance with respective applications of the present invention. Any of the patches described herein that have fixation locations may implement any of these configurations. Fixation locations 1068 of patch 30, 1030 are between two or more layers of patch 30, 1030, as described hereinabove.

For some applications, at least some of fixation locations 1068 are arranged in a plurality of segments 1070. For some applications, at least some of segments 1070 are curved when patch 30 assumes expanded shape 44. For some of these applications, at least some of the curved segments 1070 are arranged equidistantly around a center point of patch 30 when patch 30 assumes expanded shape 44. For some applications, at least some of segments 1070 are straight when patch 30 assumes expanded shape 44. For some of these applications, at least some of straight segments are arranged radially when patch 30 assumes expanded shape 44, and may not reach a center point of the patch.

Alternatively or additionally, at least some of fixation locations 768 are arranged in a plurality of points 1072. As used in the present application, including the claims, “points” are small point-like areas rather than literal zero-dimensional points.

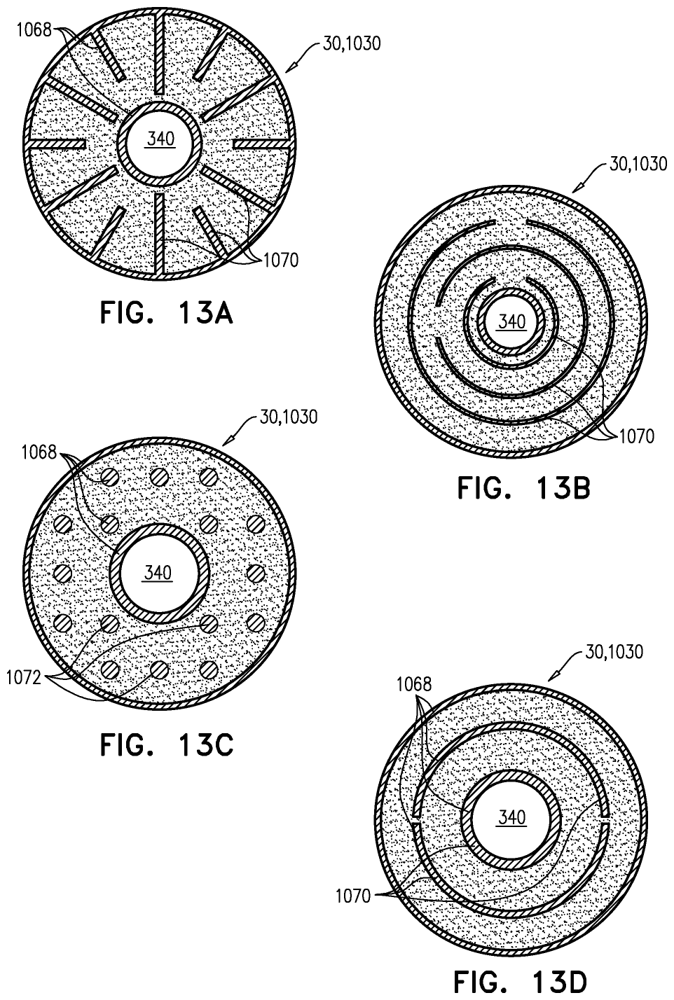

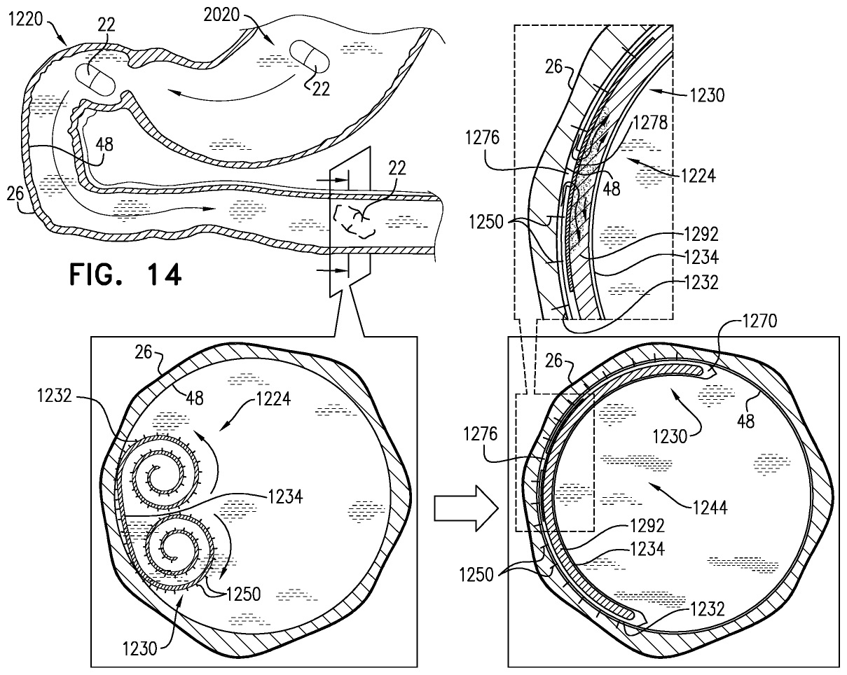

Reference is now made to FIG. 14, which is a schematic illustration of an ingestible pill 1220, for ingestion by a subject, in accordance with an application of the present invention. Ingestible pill 1220 comprises enteric coating 22, a medication 52, and a medication-delivery device 1224, which is configured to deliver medication 52.

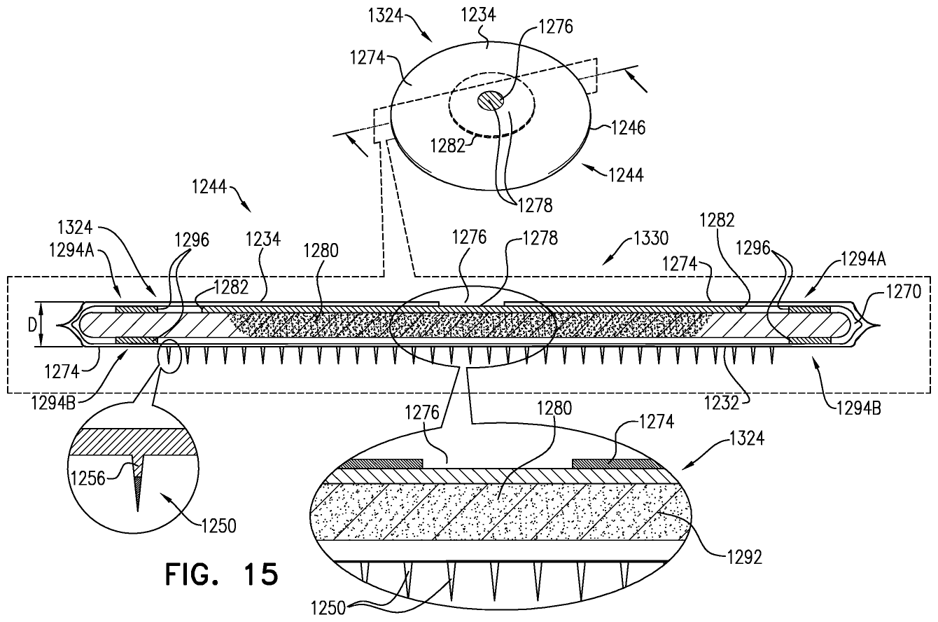

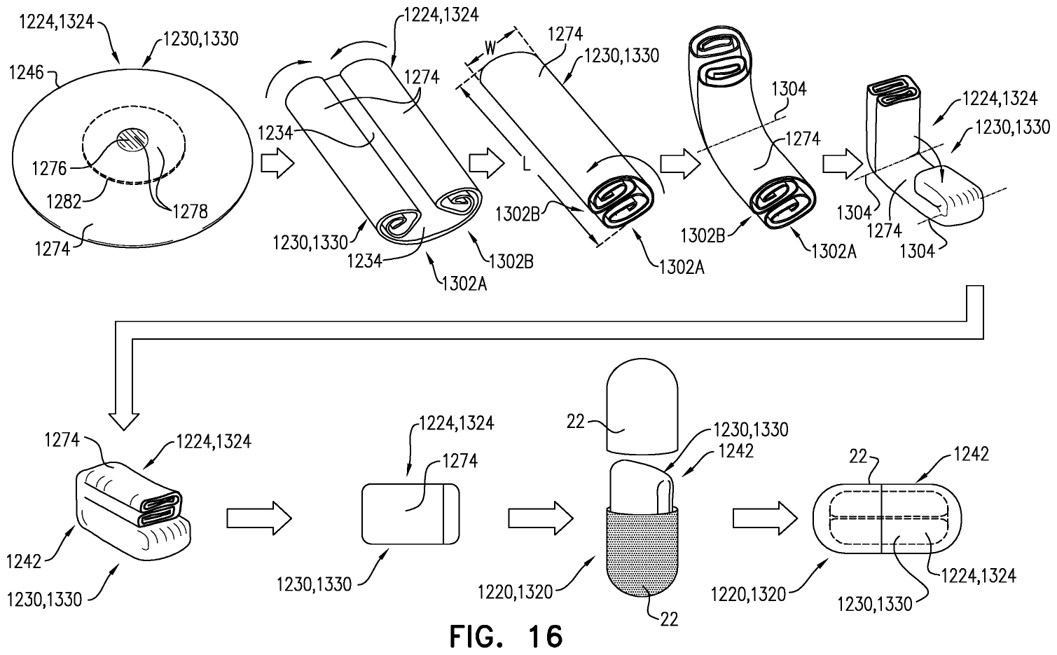

Medication-delivery device 1224 comprises a patch 1230 having an upper (intestinal-wall-contact) surface 1232 and a lower (intestinal-lumen-facing) surface 1234, which face in generally opposite directions. Patch 1230 has a compressed shape 1242 when disposed within enteric coating 22, as shown in FIG. 16, and is configured to assume, after enteric coating 22 dissolves, an expanded shape 1244, in which patch 1230 has an outer perimeter 1246 (labeled in medication-delivery device 1324, shown in FIG. 15).

Patch 1230 comprises a chamber 1270 defined by:
a substantially water-impermeable and substantially gas-impermeable, elastic layer 1274 shaped so as to define at least one window 1276 (e.g., exactly one window 1276, or more than one window 1276) therethrough; for example, elastic layer 1274 may comprise a polymer such as a thermoplastic elastomer (TPE), which may, for example, comprise thermoplastic polyurethane (TPU); polyurethane (PU); or polyethylene (PE), and
a liquid-permeable and substantially gas-impermeable wetting layer 1278, which entirely covers the at least one window 1276 and is sealed to elastic layer 1274 around the at least one window 1276; for example, wetting layer 1278 may comprise cellulose or biocellulose.

For some applications, as shown in FIGS. 14 and 15, elastic layer 1274 is shaped so as to define upper (intestinal-wall-contact) surface 1232 and lower (intestinal-lumen-facing) surface 1234 (except at the location(s) of the at least one window 1276, at which wetting layer 1278 defines the surface. Alternatively or additionally, wetting layer 1278 partially or entirely defines upper (intestinal-wall-contact) surface 1232 and/or lower (intestinal-lumen-facing) surface 1234; this configuration may increase the contact surface between wetting layer 1278 and bodily fluids, thereby increasing the efficiency of wetting.

Patch 1230 further comprises a gas-generating substance 1280, disposed within chamber 1270, that produces gas upon contact with liquid in the intestine. Wetting layer 1278 allows bodily fluids to pass into chamber 1270 and contact gas-generating substance 1280, upon dissolving of enteric coating 22 in small intestine 26. The generation of the gas within chamber 1270 promotes transition of patch 1230 from compressed shape 1242 to expanded shape 1244. For configurations in which patch is rolled and/or folded within enteric coating 22, such as described, for example, hereinbelow with reference to FIG. 16, generation of the gas within chamber 1270 may promote unrolling/unfolding of patch 1230 and/or providing structure to patch 1230 that may push upper (intestinal-wall-contact) surface 1232 against intestinal wall 48. Alternatively or additionally, for configurations in which medication-delivery device 1224 comprises medication-needles 1250 or hollow medication-delivery needles (as described hereinbelow with reference to FIGS. 14-18D), generation of the gas within chamber 1270 may push the needles against and/or into tissue of intestinal wall 48.

Typically, gas-generating substance 1280 is disposed in a vicinity of the at least one window 1276. For some applications, gas-generating substance 1280 comprises a powder. For some applications, gas-generating substance 1280 comprises sodium bicarbonate, citric acid, and/or a mixture of bicarbonate and citric acid (e.g., 60-70% sodium bicarbonate and 30-40% citric acid).

As described above, the layers described herein comprise a broad, thin, pliable material, e.g., a membrane.

As used in the present application, including in the claims, “substantially water-impermeable” and “substantially gas-impermeable” mean designed to prevent passage of water and gas, respectively, therethrough, it being understood that few materials are entirely water-impermeable or gas-impermeable.

Although window 1276 is shown as circular in the figures, this is by way of example only, and window 1276 may have any other shape, such as square, rectangular, polygonal, or elliptical. For some applications, the at least one window 1276 (e.g., exactly one window 1276, or more than one window 1276) has an aggregate area of at least 7 mm\hat{ }2, no more than 55 mm\hat{ }2, and/or between 7 and 55 mm\hat{ }2, such as between 25 and 50 mm\hat{ }2.

Elastic layer 1274 may comprise exactly one piece of material or a plurality of pieces of material (e.g., two pieces) that are sealed together to collectively define upper (intestinal-wall-contact) surface 1232 and lower (intestinal-lumen-facing) surface 1234.

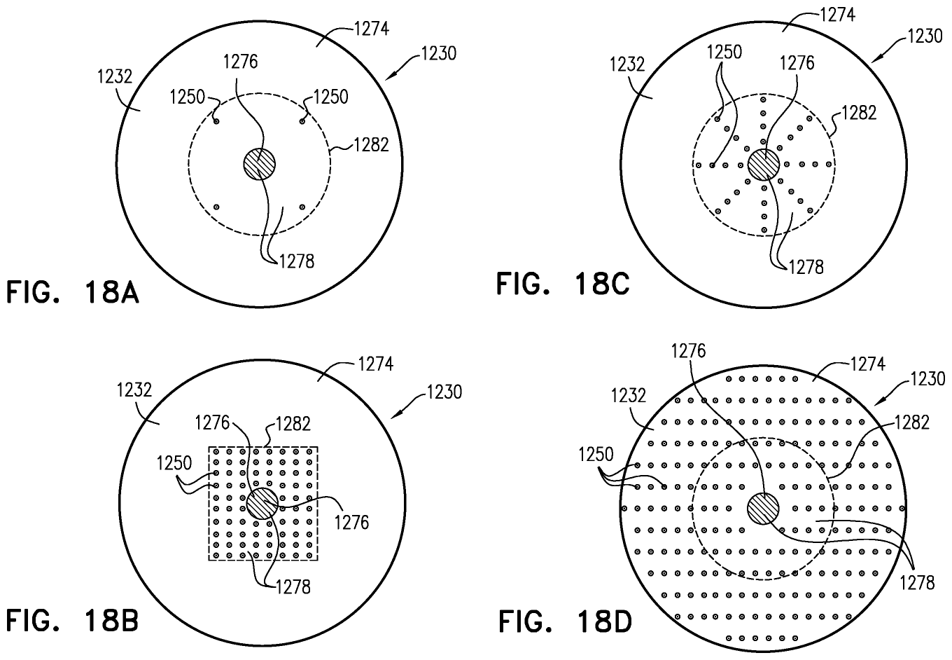

Wetting layer 1278 typically, although not necessarily, has a surface area greater than the area of window 1276, such that wetting layer 1278 overlaps with elastic layer 1274 in a region surrounding window 1276, in order to allow for good sealing of wetting layer 1278 to elastic layer 1274 around window 1276. An outer perimeter 1282 of wetting layer 1278 is shown by way of illustration and not limitation in the FIGS. 15, 16, and 18A-D. For example, wetting layer 1278 may be sealed to elastic layer 1274 by fusing (e.g., welding) or gluing. Although wetting layer 1278 is shown as circular in most of the figures, this is by way of example only, and wetting layer 1278 may have any other shape, such as square (as shown in FIG. 18C), rectangular, polygonal, or elliptical. For some applications, wetting layer 1278 is sealed to an internal surface of elastic layer 1274 around the at least one window 1276. Alternatively, wetting layer 1278 is sealed to an external surface of elastic layer 1274 around the at least one window 1276 (optionally, wetting layer 1278 entirely encapsulates patch 1230).

Typically, patch 1230 is arranged within enteric coating 22 in compressed shape 1242 (e.g., rolled, folded, or rolled and folded), such that when patch 1230 assumes expanded shape 1244 after enteric coating 22 dissolves in small intestine 26, upper surface 1232 of patch 1230 contacts small intestinal wall 48, as shown in FIG. 14. For some applications, medication-delivery device 1224 is configured to release medication 52 such that at least 60% (e.g., at least 80%, such as at least 95%) of the released medication 52 is released at upper surface 1232 of patch 1230.

Reference is still made to FIG. 14, and is additionally made to FIG. 15, which is a schematic illustration of a medication-delivery device 1324 of an ingestible pill 1320, for ingestion by a subject, in accordance with an application of the present invention.

Ingestible pill 1320 is shown in FIG. 16. Other than as described herein, ingestible pill 1320 and medication-delivery device 1324 are identical to ingestible pill 1220 and medication-delivery device 1224, respectively, and may implement any of the features of ingestible pill 1220 and medication-delivery device 1224, mutatis mutandis. Medication-delivery device 1324 comprises a patch 1330, which is identical to patch 1230, except as described below.

In medication-delivery device 1224, shown in FIG. 14, elastic layer 1274 is shaped so as to define the at least one window 1276 on an upper side of chamber 1270, such that the at least one window faces small intestinal wall 48 upon deployment. By contrast, in medication-delivery device 1324, shown in FIG. 15, elastic layer 1274 is shaped so as to define the at least one window 1276 on a lower side of chamber 1270, such that the at least one window faces away from small intestinal wall 48 upon deployment. In experiments conducted by the inventors, the inventors found that this latter orientation of the at least one window results in quicker flow of bodily fluid through the at least one window than if the at least one window faced the intestinal wall. The inventors believe that the slower flow observed when the at least one window faces the intestinal wall was caused by enteric coating 22 covering wetting layer 1278 upon transition of patch 1230/1330 to expanded shape 1244. In addition, disposing the at least one window 1276 on the lower side of chamber 1270 provides more surface area for medication 52 (e.g., medication-needles 1250) on upper surface 1232, because it is generally more difficult to dispose the medication or medication-needles on wetting layer 1278 over the at least one window. However, medication-needles 1250 may optionally be attached to wetting layer 1278, such as in configurations in which the at least one window 1276 is through upper surface 1232.

For some applications, medication-delivery device 1224/1324 further comprises a porous solid foam 1292 disposed within chamber 1270; typically, porous solid foam 1292 is shaped so as to define open pores, which allow fluid flow between pores, as is known in the foam art. Porous solid foam 1292 provides structure to medication-delivery device 1224/1324 to help maintain the device in a desired shape. For example, porous solid foam 1292 may comprise a polymer such as poly(vinyl alcohol) (PVA), polyurethane (PU), or polyethylene (PE), or silicone. As used in the present application, including the claims, the word “solid” in “solid foam” refers to the state of matter, rather than implying any level of rigidity of the foam.

For some applications, gas-generating substance 1280 is at least partially disposed in porous solid foam 1292 (i.e., within the pores of porous solid form 1292), and/or at least partially disposed between porous solid foam 1292 and wetting layer 1278.

For some applications, porous solid foam 1292 is fixed to one or more portions of an internal surface of elastic layer 1274 so as to inhibit expansion of chamber 1270 upon production of the gas by gas-generating substance 1280. This inhibition of expansion prevents chamber 1270 from becoming too spherical, thereby maintaining chamber 1270 shaped generally as a patch and/or swollen shape, e.g., swollen disc shape, when patch 1230/1330 assumes expanded shape 1244 and is unconstrained. To this end, for some applications porous solid foam 1292 is fixed to (e.g., by an adhesive 1296) at least first and second portions 1294A and 1294B of the internal surface of elastic layer 1274 that respectively define upper and lower surfaces 1232 and 1234 of patch 1230/1330.

For some applications, porous solid foam 1292 is shaped as a disc. For example, the disc may have a diameter of at least 2 cm, no more than 10 cm, and/or between 2 and 10 cm.

For some applications, porous solid foam 1292 has a volume, when dry, of at least 0.5 cm\hat{ }3, no more than 16 cm\hat{ }3, and/or between 0.5 and 16 cm\hat{ }3.

For some applications, porous solid foam 1292 has an average thickness of at least 1 mm, no more than 2 mm, and/or between 1 and 2 mm.

In some applications of the present invention, porous solid foam 1292 is integral with at least a portion of elastic layer 1274 (e.g., at least one piece of material of elastic layer 1274 for applications in which elastic layer 1274 comprises a plurality of pieces of material that are sealed together, as described hereinbelow). In other words, porous solid foam 1292 and the at least a portion of elastic layer 1274 are fabricated (e.g., molded) from a single piece of material. For example, the portion of the integral piece of material that defines porous solid foam 1292 may be shaped so as to define open pores, as described above, and the portion of the integral piece of material that defines the at least a portion of elastic layer 1274 may be shaped so as to define closed pores or no pores. For example, the integral material may comprise a polymer such as polyurethane (PU) or polyethylene (PE).

For some applications, medication 52 is solid and is shaped as at least respective portions of a plurality of medication-needles 1250 (for example, the solid medication may be integrated in a glucose structure). Typically, patch 1230/1330 is arranged within enteric coating 22 in compressed shape 1242, such that when the patch assumes expanded shape 1244 after enteric coating 22 dissolves in small intestine 26, upper (intestinal-wall-contact) surface 1232 of the patch contacts intestinal wall 48, thereby bringing medication-needles 1250 into contact with the intestinal wall.

Optionally, medication-needles 1250 further comprise respective non-medication support structures 1256. For example, non-medication support structures 1256 may serve as bases for medication-needles 1250, as shown in FIG. 15. For example, non-medication support structures 1256 may comprise a polymer, e.g., PVA or PU.

For some applications, at least 60% (e.g., at least 80%, such as at least 95%, e.g., 100%) of medication-needles 1250 are coupled to upper surface 1232 of patch 1230/1330.

For some applications, ingestible pill 1220/1320 comprises at least 4, no more than 150, and/or between 4 and 150 medication-needles 1250.

For some applications, each of medication-needles 1250 has a length of at least 20 microns (e.g., at least 50 microns), no more than 600 microns (e.g., no more than 300 microns, such as no more than 250 microns), and/or between 20 and 600 microns, e.g., between 20 and 600 microns, e.g., between 20 and 300 microns, such as between 50 and 250 microns. For some applications, an average density of all medication-needles 1250 on the surface to which medication-needles 1250 are attached is between 2 and 6 needles/mm2, such as 4 needles/mm2. Alternatively or additionally, for some applications, each of medication-needles 1250 is located within 1 mm of at least one adjacent medication-needle 1250.

For some applications, patch 1230/1330 can inscribe a circle having a diameter of at least 2 cm, less than 10 cm (e.g., less than 7 cm, such as less than 6 cm, e.g., less than 5 cm), and/or between 2 and 10 cm, e.g., between 2 and 7 cm, such as between 2 and 6, e.g., between 2 and 6 cm, when patch 1230/1330 assumes expanded shape 1244 and is unconstrained.

For some applications, a greatest distance D between upper and lower surfaces 1232 and 1234 of patch 1230/1330 is less than 6 mm, such as less than 4 mm, e.g., between 2 and 4 mm, such as between 3 and 4 mm, when patch 1230/1330 assumes expanded shape 1244 and is unconstrained (labeled in FIG. 15).

For some applications, a ratio between greatest distance D and a greatest dimension of patch 1230/1330 perpendicular to greatest distance D is less than 10%, e.g., less than 6%, such as less than 5%, and, optionally at least 1%, such as at least 2%, when patch 1230/1330 assumes expanded shape 1244 and is unconstrained.

For some applications, upper and lower surfaces 1232 and 1234 of patch 1230/1330 are substantially planar and, optionally, substantially parallel to each other, when patch 1230/1330 assumes expanded shape 1244 and is unconstrained, such as shown in FIG. 15.

Reference is now made to FIG. 16, which is a schematic illustration of a method of rolling patch 1230/1330 of medication-delivery device 1224/1324, in accordance with an application of the present invention. FIG. 16 also shows the rolled form disposed within enteric coating 22 in compressed shape 1242. For clarity of illustration, FIG. 16 shows both patch 1230 and patch 1330 schematically without needles. Patch 1230/1330 may also be rolled, folded, or rolled and folded in other ways.

In the configuration illustrated in FIG. 16, upon rolling, patch 1230/1330 is disposed within enteric coating 22 in compressed shape 1242, in which two opposite perimeter portions 1302A and 1302B of outer perimeter 1246 are (a) rolled toward each other over lower surface 1234 of patch 1230/1330 (as shown) or (b) folded toward each other over lower surface 1234 of patch 1230/1330 (not shown), such that, in either case, the two opposite perimeter portions 1302A and 1302B touch lower surface 1234, Typically, but not necessarily, patch 1230/1330 is disposed within enteric coating 22 in compressed shape 1242, in which the two opposite perimeter portions 1302A and 1302B of outer perimeter 1246 are (a) rolled at least one full turn toward each other over lower surface 1234 of patch 1230/1330 (as shown) or (b) folded at least one full turn toward each other over lower surface 1234 of patch 1230/1330 (not shown). As a result, patch 1230/1330 is rolled or folded into two separate spirals in cross section. As used in the present application, including in the claims, “folding” the patch typically does not result in sharp creases, although it optionally may.

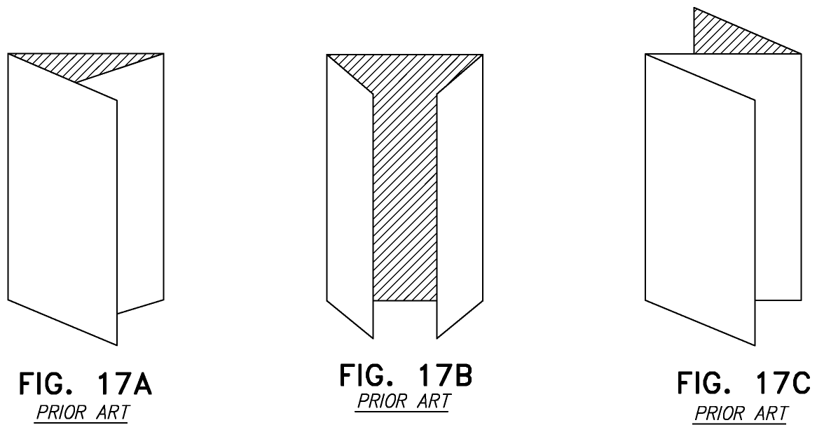

For some applications, patch 1230/1330 is disposed within enteric coating 22 in compressed shape 1242, in which:
first, the two opposite perimeter portions 1302A and 1302B of outer perimeter 1246 are (a) rolled toward each other over lower surface 1234 of patch 1230/1330 (as shown) or (b) folded toward each other over lower surface 1234 of patch 1230/1330 (not shown), such that, in either case, the rolled or folded patch 1230/1330 has a length L greater than a width W thereof (e.g., at least 2 times, such as at least 3 times greater than width W), and
then, the rolled or folded patch 1230/1330 is folded widthwise (i.e., folding about one or more lines 1304 that run in the direction of width W); optionally, the rolled or folded patch 1230/1330 is three-panel folded widthwise (as shown in FIG. 16), such as tri-folded (as shown in FIG. 16), three-panel gate folded (not shown in FIG. 16; see FIG. 17B, described below, or three-panel Z-folded (not shown in FIG. 16; see FIG. 17C, described below).

In experiments conducted by the inventors, the inventors have found that three-panel folding allows the folded patch 1230/1330 to readily unfold within a tube having a diameter of 25 mm (the approximate diameter of the human small intestine, which is typically between 25 and 30 mm), and within small intestines of pigs, because each of the folds is less than the diameter of the tube. By contrast, the inventors have found that two-panel folding, i.e., folding in half, prevents the folded patch 1230/1330 from unfolding within the 25-mm tube, because the wall of the tube blocks the unfolding. The inventors found that good unfolding and internal wall covering was achieved with a patch dimeter of between ½ and ⅔ of the perimeter of the tube/small intestine.

Reference is now made to FIGS. 17A-C, which are schematic illustrations of three-panel folds known in the art of paper folding. These three known paper three-panel folds are shown in order to define the three-panel folds described immediately above. FIG. 17A shows a paper tri-fold (also known as a three-pane rolled fold). FIG. 17B shows a paper three-panel gate fold. FIG. 17C shows a three-panel Z-fold (also known a three-panel accordion fold). As used in the present application, “three-panel folded” includes these three three-panel folds, as well as other three-panel folds not illustrated.

For some applications, patch 1230/1330 is disposed within enteric coating 22 in compressed shape 1242, in which:
first, patch 1230/1330 is rolled so as to form a single spiral in cross-section, such that the rolled patch 1230/1330 has a length greater than a width thereof (e.g., at least 2 times, such as at least 3 times greater than the width), and
then, the rolled or folded patch 1230/1330 is folded widthwise (i.e., folding about one or more lines that run in the direction of the width); optionally, the rolled or folded patch 1230/1330 is three-panel folded widthwise, such as tri-folded (see FIG. 17A), three-panel gate folded (see FIG. 17B), described above, or three-panel Z-filed (see FIG. 17C, described above)).

Reference is now made to FIGS. 18A-D, which are schematic illustrations of several configurations of patch 1230, in accordance with respective applications of the present invention. FIGS. 18A-D show upper (intestinal-wall-contact) surface 1232 of patch 1330, with respective arrays of medication-needles 1250. For example, medication-needles 1250 may be distribute over only a portion of upper surface 1232 (such as a central portion), as shown in FIGS. 18A-C, or over generally all of upper surface 1232, as shown in FIG. 18D.

Reference is made to FIGS. 14-18D. For some applications, medication-delivery device 1224/1324 comprises hollow medication-delivery needles, which are coupled to (and typically protrude from) at least one of upper surface 1232 and lower surface 1234, and are configured to deliver medication 52. In these applications, patch 1230/1330 comprises one or more medication chambers, which contains medication 52, and the hollow medication-delivery needles in fluid communication with the one or more medication chambers. For example, the one or more medication chambers may be implemented using techniques described hereinabove regarding medication chambers 670, 770, and/or 870, mutatis mutandis. The hollow medication-delivery needles may optionally have any of the characteristics, including, but not limited to, locations, number, and dimensions, of medication-needles 1250.

The inventors conducted an experiment to test a medication-delivery device similar to medication-delivery device 1224/1324, described hereinabove with reference to FIG. 16. A stomach and three meters of the small intestine were harvested from a pig. The small intestine, together with the intestinal fluid, were transferred to an operation table. The experiment was performed under X-ray observation. The medication-delivery device, in a gelatin capsule, was inserted into the small intestine using an applicator. The patch unfolded and unrolled according to design, as shown schematically in FIG. 14.

In an embodiment, techniques and apparatus described in one or more of the following applications are combined with techniques and apparatus described herein:
U.S. Pat. No. 8,287,902 to Gross;
U.S. Pat. No. 9,492,396 to Gross; and/or
U.S. patent application Ser. No. 16/103,420, filed Aug. 14, 2018.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An ingestible pill comprising:
an enteric coating;
a medication; and
a medication-delivery device, which is configured to deliver the medication and comprises a patch (a) having upper and lower surfaces that face in generally opposite directions, (b) disposed within the enteric coating in a compressed shape, (c) configured to assume, after the enteric coating dissolves, an expanded shape in which the patch has an outer perimeter, and (d) comprising:
(i) a chamber defined by:
a substantially water-impermeable and substantially gas-impermeable, elastic layer shaped so as to define at least one window therethrough, and
a liquid-permeable and substantially gas-impermeable wetting layer, which entirely covers the at least one window and is sealed to the elastic layer around the at least one window; and
(ii) a gas-generating substance that produces gas upon contact with liquid, the gas-generating substance disposed within the chamber.

2. The ingestible pill according to claim 1, wherein a greatest distance between the upper and the lower surfaces of the patch is less than 6 mm when the patch assumes the expanded shape and is unconstrained.

3. The ingestible pill according to claim 1, wherein the gas-generating substance comprises a powder.

4. The ingestible pill according to claim 1, wherein the gas-generating substance comprises sodium bicarbonate.

5. The ingestible pill according to claim 1, wherein the gas-generating substance is disposed in a vicinity of the at least one window.

6. The ingestible pill according to claim 1, wherein the wetting layer comprises a material selected from the group consisting of: cellulose and biocellulose.

7. The ingestible pill according to claim 1, wherein the patch is rolled, folded, or rolled and folded when disposed within the enteric coating in the compressed shape.

8. The ingestible pill according to claim 1, wherein the patch can inscribe a circle having a diameter of between 2 and 10 cm when the patch assumes the expanded shape and is unconstrained.

9. The ingestible pill according to claim 1, wherein the patch is configured such that the expanded shape is a swollen shape when the patch assumes the expanded shape and is unconstrained.

10. The ingestible pill according to claim 9, wherein the patch is configured such that the expanded shape is a swollen disc shape when the patch assumes the expanded shape and is unconstrained.

11. The ingestible pill according to claim 1, wherein the wetting layer is sealed to an internal surface of the elastic layer around the at least one window.

12. The ingestible pill according to claim 1, wherein the wetting layer is sealed to an external surface of the elastic layer around the at least one window.

13. The ingestible pill according to any one of claims 1-12, wherein the patch is arranged within the enteric coating in the compressed shape, such that when the patch assumes the expanded shape after the enteric coating dissolves in a small intestine of a subject, the upper surface of the patch contacts an intestinal wall.

14. The ingestible pill according to claim 13, wherein the at least one window is disposed on a lower side of the chamber.

15. The ingestible pill according to claim 13, wherein the medication-delivery device is configured to release the medication such that at least 60% of the released medication is released at the upper surface of the patch.

16. The ingestible pill according to claim 15, wherein the medication-delivery device is configured to release the medication such that at least 80% of the released medication is released at the upper surface of the patch.

17. The ingestible pill according to any one of claims 1-12, wherein the elastic layer comprises a thermoplastic elastomer (TPE).

18. The ingestible pill according to claim 17, wherein the TPE comprises thermoplastic polyurethane (TPU).

19. The ingestible pill according to any one of claims 1-12, wherein the medication-delivery device further comprises a porous solid foam disposed within the chamber.

20. The ingestible pill according to claim 19, wherein the gas-generating substance is at least partially disposed in the porous solid foam.

21. The ingestible pill according to claim 19, wherein the gas-generating substance is at least partially disposed between the porous solid foam and the wetting layer.

22. The ingestible pill according to claim 19, wherein the porous solid foam is fixed to one or more portions of an internal surface of the elastic layer so as to inhibit expansion of the chamber upon production of the gas by the gas-generating substance.

23. The ingestible pill according to claim 22, wherein the porous solid foam is fixed to at least first and second portions of the internal surface of the elastic layer that respectively define the upper and the lower surfaces of the patch.

24. The ingestible pill according to claim 22, wherein the porous solid foam is integral with at least a portion of the elastic layer.

25. The ingestible pill according to any one of claims 1-12, wherein the medication is solid and is shaped as at least respective portions of a plurality of medication-needles.

26. The ingestible pill according to claim 25, wherein the medication-needles further comprise respective non-medication support structures.

27. The ingestible pill according to claim 25,
wherein the patch is arranged within the enteric coating in the compressed shape, such that when the patch assumes the expanded shape after the enteric coating dissolves in a small intestine of a subject, the upper surface of the patch contacts an intestinal wall,
wherein at least 60% of the medication-needles are coupled to the upper surface of the patch.

28. The ingestible pill according to claim 27, wherein at least 80% of the medication-needles are coupled to the upper surface of the patch.

29. The ingestible pill according to claim 27, wherein the at least one window is through the lower surface of the patch.

30. The ingestible pill according to claim 25, wherein each of the medication-needles has a length of between 20 and 600 microns.

31. The ingestible pill according to claim 25, wherein a greatest radius of the medication-needles is between 20 and 150 microns.

32. The ingestible pill according to any one of claims 1-12, wherein the medication-delivery device comprises hollow medication-delivery needles, which are configured to deliver the medication.

33. An ingestible pill comprising:
an enteric coating;
a medication; and
a medication-delivery device, which is configured to deliver the medication and comprises a patch having upper and lower surfaces that face in generally opposite directions, and an outer perimeter,
wherein the patch is disposed within the enteric coating in a compressed shape, in which two opposite perimeter portions of the outer perimeter are (a) rolled toward each other over the lower surface of the patch or (b) folded toward each other over the lower surface of the patch, such that, in either case, the two opposite perimeter portions touch the lower surface, and
wherein the patch is configured to assume an expanded shape after the enteric coating dissolves.

34. An ingestible pill comprising:
an enteric coating;
a medication; and
a medication-delivery device, which is configured to deliver the medication and comprises a patch having upper and lower surfaces that face in generally opposite directions,
wherein the patch is disposed within the enteric coating in a compressed shape, in which the patch is three-panel folded, and
wherein the patch is configured to assume, after the enteric coating dissolves, an expanded shape in which the patch has an outer perimeter.

35. A method comprising:
receiving, by a subject, an ingestible pill, which includes an enteric coating; a medication; and a medication-delivery device, which is configured to deliver the medication and includes a patch (a) having upper and lower surfaces that face in generally opposite directions, (b) disposed within the enteric coating in a compressed shape, and (c) including: (i) a chamber defined by: (A) a substantially water-impermeable and substantially gas-impermeable, elastic layer shaped so as to define the upper and at least one window therethrough, and (B) a liquid-permeable and substantially gas-impermeable wetting layer, which entirely covers the at least one window and is sealed to the elastic layer around the at least one window; and (ii) a gas-generating substance that produces gas upon contact with liquid, the gas-generating substance disposed within the chamber; and
swallowing the ingestible pill by the subject, so that the coating dissolves in a small intestine and the patch assumes an expanded shape, in which the patch has an outer perimeter.

36. A method comprising:
receiving, by a subject, an ingestible pill, which includes an enteric coating; a medication; and a medication-delivery device, which is configured to deliver the medication and includes a patch having upper and lower surfaces that face in generally opposite directions, and an outer perimeter, wherein the patch is disposed within the enteric coating in a compressed shape, in which two opposite perimeter portions of the outer perimeter are (a) rolled toward each other over the lower surface of the patch or (b) folded toward each other over the lower surface of the patch, such that, in either case, the two opposite perimeter portions touch the lower surface; and
swallowing the ingestible pill by the subject, so that the coating dissolves in a small intestine and the patch assumes an expanded shape.

37. A method comprising:
receiving, by a subject, an ingestible pill, which includes an enteric coating; a medication; and a medication-delivery device, which is configured to deliver the medication and includes a patch having upper and lower surfaces that face in generally opposite directions, wherein the patch is disposed within the enteric coating in a compressed shape, in which the patch is three-panel folded; and
swallowing the ingestible pill by the subject, so that the coating dissolves in a small intestine and the patch assumes an expanded shape in which the patch has an outer perimeter.

38. An ingestible pill comprising:
an enteric coating; and
a medication-delivery device, which includes (a) a patch having upper and lower surfaces that face in generally opposite directions; and (b) needles,
wherein the patch is disposed within the enteric coating, folded so as to define one or more creases, which define respective inner and outer crease sides, wherein at least 50% of the needles are coupled to the patch along the inner crease sides, and
wherein the patch is configured to assume, after the enteric coating dissolves, an expanded shape, in which the patch has an outer perimeter.
